(19) United States
(12) Patent Application Publication
(10) Pub. No.: US 2025/0066849 A1
(43) Pub. Date: Feb. 27, 2025

(54) Anodized Metal Oxide Substrates Supporting Solid Support Feature Arrays for Patterned Flowcells

(71) Applicant: Illumina, Inc., San Diego, CA (US)

(72) Inventor: Timothy Beerling, Del Mar, CA (US)

(73) Assignee: Illumina, Inc., San Diego, CA (US)

(21) Appl. No.: 18/760,831

(22) Filed: Jul. 1, 2024

Related U.S. Application Data
(60) Provisional application No. 63/534,259, filed on Aug. 23, 2023.

(51) Int. Cl. C12Q 1/6874; B01L 3/00; C12Q 1/6804

(52) U.S. Cl. CPC C12Q 1/6874; B01L 3/502761; C12Q 1/6804; B01L 2200/0647; B01L 2200/16; B01L 2300/0654; B01L 2300/0819; B01L 2300/0877; B01L 2300/168

(57) ABSTRACT

A flow cell assembly is provided that includes a support layer of low-background material, a film of anodized metal oxide (AMO) material adhered to the support layer, and a substrate formed, in part, by one or more patterns imparted in the AMO material. A flow cell assembly herein may include a support layer of low-background material, a film of AMO material adhered to the support layer, and a substrate surface comprising one or more patterns imparted in the AMO material. Each pattern may comprise an array of nanowell features surrounded by interstitial regions of featureless AMO film.

### CROSS-REFERENCE TO RELATED APPLICATIONS

This application claims priority to U.S. Provisional Application No. 63/534,259, filed Aug. 23, 2023, the entire disclosure of which is hereby incorporated by reference herein in its entirety.

### BACKGROUND

Next Generation Sequencing (NGS) and other high-throughput sequencing workflows and corresponding systems exploit a complex collection of technologies. Improvements in materials use, computational leveraging, and processing efficiency have been shown to provide significant benefits in terms of data output volume, run times, and cost effectiveness. However, given the interdependency of the many technologies in the sequencing systems, the challenge is to develop cost improvements without adversely impacting the quality of read data and reliability of downstream analyses such as read mapping and genome assembly.

For illustration, sequencing methodologies for nucleic acid materials on NGS platforms commonly deploy deoxyribonucleic acid (DNA) libraries in which a DNA target (e.g., genomic DNA (gDNA), or complimentary DNA (cDNA)) is processed into fragments and ligated with technology-specific adaptors. NGS workflow using, e.g., a sequence-by-synthesis (SBS) technique, involves loading a DNA library onto a flow cell and hybridizing individual DNA fragments to adapter-specific complimentary oligonucleotides (oligos) covalently bound to the flow cell surface (planar or patterned); clustering the individual fragments into thousands of identical DNA template strands (amplicons) through amplification (e.g., bridge or exclusion); and, finally, sequencing, in which copy strands are simultaneously synthesized and sequenced on the DNA templates using a reversible terminator-based process that detects signals emitted from fluorophore-labeled single bases as they are added round by round to the copy strands. Because the multiple template strands of each cluster have the same sequence, base pairs incorporated into the corresponding copy strands in each round will be the same, and thus the signal generated from each round will be enhanced proportional to the number of copies of the template strand in the cluster.

Flow cells provide a convenient format for high throughput sequencing operations involving multiple cycles of repeated chemical delivery and image capture. The density at which clusters can be packed onto a flow cell and the speed at which a flow cell can be imaged generally determine the throughput and cost of sequencing. For patterned flow cells, nanowells are etched into the substrate surface for optimal cluster spacing. The more densely the nanowells can be etched onto the flow cell, the greater the sequencing output per flow cell and reagent kit, and the more cost-effective the sequencing operation. For example, current patterned flow cells can support a nanowell array having a 1:1 pitch ratio of nanowell diameter and shortest (or nearest neighbor) distance between adjacent nanowells. However, nanowell (and cluster) density is extrinsically limited both to a relative smallest near neighbor distance determined by the resolving power of the particular optical instrument and the absolute smallest near neighbor distance imposed by the Abbe diffraction limit equal to half the wavelength of emitted light (at a Numerical Aperture (NA) value of 1).

High quality detection lenses and other optical components may be implemented to increase resolving power, including use of high Numerical Aperture (NA) objective lenses and sensor enhancements such as charge-coupled device (CCD) implementing time delay integration (TDI). However, such implementations come with tradeoffs. For example, introduction of a high NA objective, results in a reduction of the available Depth of Focus (DoF). Therefore, local variations in tilt or height among nanowells of a subject tile may result in excursions of portions of the sample outside of the DoF, resulting in defocused image portions and a reduction in data quality. Moreover, CCD camera implementations may be sensitive to variations in nanowell spacing. Although the approximate spacing of a nanowell array may be known, the precise location of a given well in relation to neighboring wells is not. Thus, when a CCD camera is positioned directly adjacent to the faceplate of a subject tile, the wells may not be evenly distributed along the pixels of the CCD camera and, as such, the wells will not be aligned in a known manner with the pixels. This misalignment can result in spatial crosstalk between adjacent wells, which, in turn, can degrade data quality of the particular signal of interest.

The further downscaling of materials requires that nanoelements with precise and uniform shape and size are synthesized, positioned at a desired location and organized into two- or three-dimensional architectures. While optical lithography is reaching its resolution limit, current nanolithographic techniques (electron-beam, X-ray, or focused ion beam) are expensive and/or have a low throughput, which makes their use in industrial environments impractical. For these and other reasons that will become apparent from the present disclosure, there remains a need for enhanced nanoconstruction of patterned arrays that can generate high quality data from signals of interests with enhanced cost efficiency.

### SUMMARY

The present disclosure relates to nanoporous metal oxide materials as templates for the controlled growth of solid support nanostructures for molecular analyses of biological materials. In particular, metal oxide films subject to anodic oxidation (anodized metal oxide (AMO)) form pores that self-assemble under electrochemical bias into ordered arrays of close-packed hexagonal nanowells. Fabrication techniques herein may be deployed for large-scale, cost-efficient production of high throughput nanodevices for evaluation of biological materials with high accuracy.

Various example embodiments herein are directed nanodevice assemblies incorporating patterned AMO films, as well as methods and systems implementing such AMO-based nanodevices in connection with molecular analysis of a biopolymeric sample, including high-throughput sequencing of nucleic acid materials on NGS and other analytic platforms based in fluorescence microscopy. Nanodevices as contemplated herein include any assay platform implementing a patterned solid support substrate for analyzing biological analytes, e.g., flow cells, microarrays, and bead chips.

In some examples, a pattern etched in the AMO material may be an array of nanowells surrounded by interstitial regions of featureless or planar AMO film. In the example, each nanowell includes an opening formed in the AMO material, an interior volume having an average depth at least equal to a thickness of the interstitial region surrounding it, and a base comprising an exposed surface of the low-background material of the support layer, which may serve as a solid support for detection of a biological sample. In some examples, two or more patterns in an x-y format as lanes are arranged axially along a y-axis of the substrate surface for flowing a chemical solution (e.g., biological sample or reagents) across solid support or other reaction site structures collocated with the patterned array. The lane patterns may be radially separated along the x-axis of the substrate surface by a noncontiguous masking layer bonded to the AMO film, forming a fluidic channel system typical of flow cells suited to molecular analysis as described herein.

In one embodiment, a flow cell assembly is provided that includes a support layer of low-background material, a film of AMO material adhered to the support layer, and a substrate formed, in part, by one or more patterns imparted in the AMO material. In one such embodiment, a flow cell assembly herein may include a support layer of low-background material, a film of anodized metal oxide (AMO) material adhered to the support layer, and a substrate surface comprising one or more patterns imparted in the AMO material. Each pattern may comprise an array of nanowell features surrounded by interstitial regions of featureless AMO film. Each nanowell feature may be characterized by an interior volume, an opening formed in the AMO material, and a base. The interior volume may have an average depth at least equal to a thickness of an interstitial region surrounding each respective feature. In that configuration, the base may include an exposed surface of low-background material of the support layer. The exposed surface of low-background material may be functionalized as a solid support for detection of a biological sample.

The AMO film may be processed from any suitable metal oxide (including dioxides and trioxides) that forms natural porous structures under anodic oxidation as described herein, including, e.g., aluminum oxide anodized to anodic aluminum oxide (AAO), and titanium oxide anodized to anodic titanium oxide (ATiO), hafnium oxide (HfO2) anodized to anodic hafnium oxide (AHO), tantalum oxide anodized to anodic tantalum oxide (ATaO), niobium oxide anodized to anodic niobium oxide (ANO), cerium oxide anodized to anodic cerium oxide (ACO), and gallium oxide anodized to anodic gallium oxide (AGO), tungsten oxide anodized to anodic tungsten oxide (AWO), zirconium oxide anodized to anodic zirconium oxide (AZO), and tin oxide anodized to anodic tin oxide (ASnO). The support layer may be any suitable low-background material, including materials exhibiting both high transmissivity and high fluorescence transparency, particularly for use as solid supports for fluorescence-based imaging implementations. Suitable low-background materials for use as support layers includes, e.g., glass, silica, organo-silicate, polymers, amorphous metals, and quartz.

In some examples, solid support structures are functionalized with a surface chemistry that enables various operations of an NGS workflow, including immobilization, amplification, and synthesis operations (e.g., sequencing-by-synthesis (SBS)). For example, solid support structures herein may be coated with an adhesion promoter such as silane. The solid support may also include an intermediate polymer layer for covalently binding or otherwise hosting capture agents used to immobilize target nucleic acid materials to the solid support. In one example in which an exposed surface of the low-background material is functionalized as a solid support, as described above, the exposed surface may be silanized. In one such example, the low-background material is a glass and the exposed surface of the glass functionalized as a solid support is a silanized glass surface. The silanized glass surface may also include a polymer layer contiguous with the silanized glass surface and a surface chemistry grafted to the polymer layer, which is selected for interaction with a biological sample. In one example, the surface chemistry comprises one or more primers adapted to interact with one or more constituent analytes of a biological sample.

Variations in AMO patterning including the diameter, shape, depth, and pitch of the naturally forming nanowell arrays may be controlled through the addition or manipulation of various electrochemical and other processing conditions. Surface morphology of AMO films herein may thus be calibrated to the resolving power of optical components implemented in a particular optical sequencing system. In the context of NGS workflows, for example, nanowells may be configured to have an average diameter between about 250 nm and 400 nm, an average pitch between about 350 nm and 750 nm, and an average depth between about 200 nm and 400 nm. Moreover, each such nanowell may also provide a functionalized, low-background solid phase support for signal detection of constituent polynucleotide analytes of a sample nucleic acid material at appropriately high SNR values.

In some examples, the natural hexagonal morphology of the nanowell array is retained. In other examples, the nanowell array in the AMO film is reconfigured as cylindrical structures with substantially circular openings. Nanowell morphology may be calibrated to the particular operation, support format, or imaging equipment for a given molecular analysis, wherein the openings of nanowells are substantially circular. In one example, a flow cell implemented on an analytic platform based in fluorescence microscopy, the average diameter (cylindrical configurations) or average long diagonal distance (hexagonal configurations) of a nanowell array may be about 250 nm or greater, 300 nm or greater, 350 nm or greater, 400 nm or greater, 450 nm or greater, or 500 nm or greater, or may be in a range between about 250 nm and 600 nm, 300 nm and 500 nm, or 350 nm and 450 nm. In another example, the average pitch may about 250 nm or greater, 300 nm or greater, 350 nm or greater, 400 nm or greater, 450 nm or greater, 500 nm or greater, 550 nm or greater, 600 nm or greater, 650 nm or greater, or 700 nm or greater, or may be in a range between about 250 nm and 800 nm, 300 nm and 750 nm, 350 nm and 700 nm, 400 nm and 650 nm, 450 nm and 600 nm, 500 nm and 550 nm. In another example, the average depth of the nanowell array is 150 nm or greater, 200 nm or greater, 250 nm or greater, 300 nm or greater, 350 nm or greater, or 400 nm or greater, or may be in a range between about 150 nm and 500 nm, 200 nm and 450 nm, or 250 nm and 400 nm, or 300 nm and 350 nm.

In one aspect, biopolymeric assays provide a biopolymeric sample absorbed on patterned substrate imparted in AMO material. In certain such embodiments, a biopolymeric assay includes a flow cell that incorporates a support layer of low-background material, a cladding layer of AMO material, and a substrate surface having one or more patterns etched in the AMO material. Each pattern may include an array of nanowell features surrounded by interstitial planar regions of the cladding layer. Each nanowell in the array may include (a) an interior volume having a depth at least equal to the thickness of the interstitial region surrounding it, (b) an opening formed in the cladding layer, and (c) a bottom opposite the opening, where the bottom and interior of the nanowell are in fluid communication with the substrate surface via the opening. The bottom may include a solid phase support formed by an exposed surface of the low-background material of the support layer.

In some examples, immobilization of the biopolymeric sample onto solid supports is affected through sample-specific primers. The biopolymeric sample may take the form of constituent analytes of a subject biopolymeric material, and each may be ligated with a primer-specific moiety, in which case sample-specific primers may be adapted to interact with the primer-specific moiety. In one example embodiment of a polynucleotide assay, the primer-specific moiety is a nucleotide sequence capable of hybridizing with a complementary sequence of the sample-specific primers. In other embodiments, immobilization may be affected through interaction between capture agent conjugates of constituent analytes and complementary binding partners coated on solid support surfaces. For some such embodiments, biotin and avidin (or streptavidin) moieties serve as respective binding agents and partners. In still other embodiments, immobilization may be affected through click chemistry using, e.g., clickable groups such as azides or linear or cyclic alkynes.

In one example embodiment, a biopolymeric assay includes a flow cell functionalized with a surface chemistry on which a biopolymeric sample is immobilized. The flow cell may include support layer of low-background material, a cladding layer of anodic alumina oxide (AAO) material, and a substrate surface having one or more patterns etched in the AAO film. The one or more patterns of the AAO film may include, e.g., an array of nanowell features surrounded by interstitial planar regions of the cladding layer, where each feature include an opening formed in the cladding, a bottom opposite the opening and an interior volume, where the bottom and interior of the nanowell are in fluid communication with the substrate surface via the opening. The interior volume may have a depth at least equal to a thickness of an interstitial region surrounding the respective feature, in which case the bottom comprises a solid support formed of an exposed surface of the low-background material of the support layer. The surface chemistry of the biopolymeric assay may include one or more primers grafted to each solid support of at least a portion the array of nanowell features, where each primer includes a capture moiety adapted to interact with a constituent analyte of a biopolymeric sample. For example, an interaction between the capture moiety and a constituent analyte may result in the constituent analyte being immobilized on a respective solid support. The biopolymeric sample may include any of a variety of polynucleotides or polypeptides. For example, the biopolymeric material may include a nucleic acid material in which each constituent analyte is a DNA fragment of the nucleic acid material and the capture moiety comprises a complementary sequence of nucleotides.

In one aspect, methods implementing patterned substrates and biopolymeric assays herein are provided. Such methods generally may include providing a patterned substrate incorporating an array of nanowells imparted in an AMO film and a solid phase support of low-background material collocated with the nanowell array; grafting an appropriate surface chemistry to the solid phase support at each collocation; contacting a biopolymeric sample with the solid phase support; and optically detecting constituents of the biopolymeric sample.

Methods herein are well-suited to a variety of optical detection workflows and systems. In some examples, optical detection methods may be performed in connection with sequencing-by-synthesis (SBS), cyclic-array sequencing, pyrosequencing, and various other sequencing methodologies in NGS workflows. In other examples, optical detection methods may be performed in connection with genotyping systems implementing single nucleotide polymorph (SNP), copy number variant (CNV), and other genotyping assays. To that end, patterned substrates may be supported on a BeadChip, microarray, or other local support typically used in connection with genotyping assays, and as disclosed herein. In addition, optical detection methods herein may be performed in systems implementing various microscopy techniques, including, e.g., fluorescent imaging, epi-fluorescent imaging, and total-internal-reflectance-fluorescence (TIRF) imaging, super resolution imaging, including structured illumination microscopy (SIM), and subpixel imaging using, e.g., time-delay integration (TDI).

In one example embodiment, a method of detecting a nucleic acid sample includes providing a flow cell having a support layer of a low-background material, a film of anodized metal oxide (AMO) material adhered to the support layer, and a substrate surface with one or more patterns etched in the AMO material. Each pattern may include an array of nanowell features surrounded by interstitial regions of featureless AMO film, where an interior volume of each nanowell has a depth at least equal to a thickness of a surrounding interstitial region of a respective feature. The nanowell features may also include an opening formed in the AMO material, and a bottom opposite the opening, where the bottom and interior of the nanowell are in fluid communication with the substrate surface via the opening, and the bottom comprises a solid support formed of an exposed surface of the low-background material of the support layer. The method may further include grafting a surface chemistry to each solid support of at least a portion of the array of nanowell features, where the surface chemistry may include a one or more primers grafted to a respective solid support, and each primer is configured as or with a capture moiety adapted to interact with a constituent analyte of the nucleic acid sample.

The method may still further include one or more of: processing the nucleic acid sample into a pool of fragment polynucleotide analytes in solution; flowing the solution on the substrate surface; contacting at least a portion of the pool of constituent polynucleotide analytes with at least a portion of the array of nanowell features such that at least one constituent polynucleotide analyte of the portion of the pool is immobilized on one solid support of the portion of the array; and detecting a subject analyte of the immobilized constituent analytes.

In certain examples, a fluorescence microscopy technique is used for detecting a subject analyte of the immobilized constituent analytes. In that regard, the method may include providing an optical detection system having an excitation source, one or more optical sensors, and a signal processor. In one such example, the detection of a subject analyte may include the steps of: irradiating, by the excitation source, the subject analyte with an excitation light, where the irradiation of the subject analyte causes emission of an optical signal from the subject analyte; detecting, by the one or more optic sensor, the optical signal; obtaining from the optical signal, by the signal processor, data indicative of a characteristic of the subject analyte, e.g., a base type of a nucleotide of a subject polynucleotide analyte.

Systems, methods, and devices implementing AMO-based substrates may also incorporate additional detection modalities. For example, an AMO-based substrate may further support an electrode array for generating a current in response to chemical interactions involving a biological sample. An AMO-based substrate may further support an array of biosensors forming one or more waveguides for evanescent fluorescence detection of a biological sample.

Optical imaging systems implementing patterned substrates in accordance with these and other embodiments generate high quality data from signals of interests with enhanced cost efficiency.

### BRIEF DESCRIPTION OF THE DRAWINGS

FIG. 1A is a top view of an example flow cell;

FIG. 1B is an enlarged, and partially cutaway view of an example flow cell architecture and functionalized solid support;

FIGS. 1C and 1D are schematic cross-sectional views that show composite structures and assemblies of an example flow cell.

FIGS. 2A and 2B are a schematic illustrations of example nanowell features in a bead chip implementation.

FIG. 3 is a block diagram of an example process for preparing an example flow cell of FIG. 1.

FIGS. 4A-D are schematic cross-sectional views of, in 4A, an example intermediate composite structure of deposition process of FIG. 3, and, in 4B, an example intermediate composite structure of anodization process (or processes) of FIG. 3 as applied to the intermediate structure of the deposition process, and, in 4C, an example intermediate composite structure of an imprinting/masking process of FIG. 3 and, in 4D, an intermediate composite structure of an anodization process (or processes) of FIG. 3 as applied to the intermediate structure of the imprinting/masking process.

FIG. 5 is a schematic illustration of example hexagonal AMO nanostructures and measurement references for pore diameter, cell diameter, and pitch.

FIG. 6 is a schematic illustration of an example intermediate anodization process of FIG. 3.

FIGS. 7A-C are graphs plotting, in 7A, applied voltage to cell/pore diameter and porosity, in 7B, acidic electrolyte concentration to current density and AMO (AAO) growth rate, and 7C current densities over time under respective potentiostatic or galvanostatic conditions.

FIG. 8 are schematic cross-sectional views of example intermediate composite structures of respective silanization (a) and hydrogel spin coating/linking (b) steps of a finishing process of FIG. 3 and an example finished composite structure of a polishing step (c) of a finishing process of FIG. 3

FIG. 9 illustrates fiducial examples.

FIG. 10 is a schematic cross-sectional view of an example of an electrode assembly incorporated in a nanowell feature of the flow cell of FIG. 1.

### DETAILED DESCRIPTION

Various protocols in biomolecular research involve performing a large number of controlled reactions on local support surfaces or within predefined reaction chambers. The desired reactions may then be observed or detected, and subsequent analysis may help identify or reveal properties of a chemical involved in the reaction. Many such protocols involve analyses of biopolymeric materials including nucleic acid and polypeptide sequencing, genotype screening, loss-of-function analysis, and genetic association analysis, to name just a few. For example, a variety of nucleic acid sequencing techniques utilize optically detectable samples and/or nucleoside reagents on solid phase supports, including, for illustration herein, sequencing-by synthesis (SBS). Other methodologies utilize probe-grafted arrays for screening biopolymeric materials for a locus of interest, including, e.g., single nucleotide polymorph (SNP) screens. These techniques are particularly well suited to the systems, methods and devices of the present disclosure and therefore highlight various advantages for particular embodiments herein.

SBS techniques implemented, e.g., on an NGS platform, typically sequence nucleic acid materials processed as DNA libraries, in which a DNA target or a cDNA of an RNA target is enzymatically fragmented into constituent analytes (or fragments) and ligated with technology-specific adaptors. NGS workflow generally involves loading the DNA library onto a flow cell and hybridizing individual DNA fragments to adapter-specific complimentary oligonucleotides (surface oligos) covalently bound to the flow cell surface; and clustering the individual fragments into thousands of identical DNA template strands (sometimes referred to as amplicons) through amplification. For nanowell arrays, only the solid state surface of each nanowell are functionalized with surface oligos, and DNA library fragments are seeded within individual nanowells, ideally in a 1:1 fragment to nanowell ratio, and then clustered, resulting in monoclonal single-stranded DNA template populations discretely contained within each nanowell and spaced apart by blank interstitial space.

SBS proceeds in a series of cycles, in which copy strands are simultaneously synthesized and sequenced on DNA templates in parallel using a reversible terminator-based process that detects signals emitted from fluorescently-labeled single nucleosides as they are added cycle by cycle to the copy strands. In a given cycle, single labeled nucleosides are added to copy strands in parallel, labeled nucleosides added in the cycle are illuminated to induce fluorescence, signals emitted by fluorescing nucleosides are detected, and base calls are made through signal processing. Imaging of the array during the cycle is generally performed iteratively in sections (or tiles) within the field of view of the imager. Upon completion of a cycle, the labeled nucleosides are removed and replaced by non-labeled analogues and sequencing proceeds to a next sequencing cycle.

In addition to SBS, various implementations herein have application to any number of other sequencing techniques, including, e.g., cyclic-array sequencing, real time sequencing; nanopore sequencing; long read sequencing; single-molecule sequencing; stochastic sequencing; amplification-free sequencing; sequencing by ligation; pyrosequencing; and ion semiconductor sequencing.

Systems, methods, and devices herein may also utilize probe-grafted arrays for screening biological molecules, such as nucleic acids and polypeptides, for a locus of interest. Such microarrays may include deoxyribonucleic acid (DNA) or ribonucleic acid (RNA) capture probes, which are specific for nucleotide sequences present in humans and other organisms. In certain applications, for example, individual DNA or RNA probes may be grafted at addressable reaction sites on an array surface. A test sample, such as from a known person or organism, can be exposed to the array, such that target nucleic acids hybridize to complementary probes grafted on the array. The probes can be labeled in a target specific process (e.g., due to labels present on the target nucleic acids or due to enzymatic labeling of the probes or targets that are present in hybridized form). The array can then be examined by scanning specific frequencies of light over the analytes to identify which target nucleic acids are present in the sample.

In an example, a genotyping device may receive the microarray and perform the scanning of the frequencies of light over the analytes to generate image data comprising raw images or signals that may be processed to identify target nucleic acids. A genotyping application as contemplated may be implemented to screen for the presence of a genetic locus of interest in a target nucleic acid sample. A locus of interest in a typical genotyping protocol, and as disclosed herein, may include, without limitation, polymorphs (e.g., single nucleotide polymorphs (SNPs), indels), short tandem repeats (STR), copy number variants (CNV), germline variants, methylation sites (e.g., CpG islands), and exogenous sequences (e.g., virus). Target nucleic acid samples herein may include polynucleotides of any length, and may be derived from any number of genetic sources including from human or non-human organisms, and from individual organisms or organism populations. Samples herein may be obtained from wide variety of genetic materials—e.g., gDNA, mtDNA, mRNA, cDNA transcribed from mRNA, non-coding RNA, and small RNA, polynucleotide conjugates, analogues, and amplicons.

Any of a variety of array configurations (also referred to as “microarrays”) known in the art can be used in a system, method or device set forth herein, including, e.g., with assay workflows for SNP genotyping. Image-generating chip arrays provide a convenient format for assaying SNPs, particularly at commercial scale. An example workflow may begin with accession and extraction of a DNA sample, either from single cell source or a tissue sample. The extracted DNA sample may be amplified, usually off-chip in solution, and the amplicon output is then subjected to controlled enzymatic fragmentation. The processed DNA sample is loaded onto the image-generating chip and subjected to hybridization using locus specific oligo probes functionalized on the chip substrate. Allelic specificity of hybridized DNA is conferred by enzymatic base extension at 3′ end of the probe. Base extensions are applied fluorescent labels, imaged under excitation, and allele signal intensity data is used to perform genotype calling. An array may be functionalized with individual probe or a population of probes. In the latter case, the population of probes at each analyte is typically homogenous having a single species of probe. For example, in the case of a nucleic acid array, each locus specific probe may be amplified to yield multiple nucleic acid molecules each having a common sequence. However, in some implementations the population of probes at a given reaction site of an array can be heterogeneous. Similarly, protein arrays can be functionalized with a single protein probe or a population of protein probes typically, but not always, having the same amino acid sequence. The probes can be attached to the surface of an array for example, via covalent linkage of the probes to the surface or via non-covalent interaction(s) of the probes with the surface.

Various types of fluorescence microscopy may be used with embodiments described herein. Fluorescence microscopy is performed using an optical sequencing system that includes a light source (e.g., lasers, light emitting diodes (LEDs)) tuned to wavelengths of light that induce excitation in the fluorescent dyes used for labelling a sample biopolymeric material or probe; one or more optical instruments, such as cameras, lenses, sensors, to capture signals emitted through induced excitation, and one or more processors for developing composite images from captured signals emitted from labelled targets within the optical elements' field of view (tile) in a given sequencing assay. For example, embodiments may be configured to perform at least one of conventional fluorescent imaging, epi-fluorescent imaging, total-internal-reflectance-fluorescence (TIRF) imaging, a time-delay integration (TDI) imaging (CCD-TDI or CMOS-TDI), or Super Resolution imaging, e.g., Structured Illumination Microscopy (SIM). Furthermore, the imaging sessions may include “line scanning” one or more samples such that a linear focal region of light is scanned across the sample(s). Imaging sessions may also include moving a point focal region of light in a raster pattern across the sample(s). Alternatively, one or more regions of the sample(s) may be illuminated at one time in a “step and shoot” manner.

Systems, methods, and devices herein may be used in connection with a number of high-resolution imaging systems and techniques. Optical imaging systems may be limited by the optical resolution of the data capable of being detected by the optical components of the system. In microscopy, optical resolution is the shortest distance between two separate points in a microscope's field of view that can still be distinguished as distinct entities, i.e., the Rayleigh limit. For example, the optical resolution of such objects may be expressed as a function of a wavelength (λ) of light in the optical sequencing system, in which shorter wavelengths yield higher resolution, and an objective, or optical element (e.g., lens or lenses) used to gather the light from the target objects, which may be measured by a numerical aperture (NA). NA of an objective lens is given by the formula nsine θ. where n is the index of refraction of the medium in which the lens is working (nair≈1), and θ is the half-angle of the maximum cone of light that can enter or exit the lens.

In one example, high-resolution images may be obtained through an imaging system incorporating a high NA objective. An optical sequencing system using an objective lens having a relatively high NA can resolve more closely adjacent point sources compared to a system characterized by a relatively lower NA. NA thus determines the resolving power of an objective lens of an optical sequencing system. The higher the NA of the total system, the better the resolution. Higher quality detection lenses and other detection optical elements thus may be used to improve the optical resolution of the optical sequencing systems.

In another example, high-resolution images may be obtained through a subpixel imaging system, e.g., TDI using CCD- or CMOS-based sensors. Subpixel imaging is based on increasing a sample rate to raise the Nyquist frequency, which limits the highest frequency the optical sequencing system can reliably measure (e.g., translate digitally) to one half the sample rate at which the equipment operates. Subpixel imaging is performed by staggering TDI sensors by a subpixel offset and subsampling a given collection area, which effectively doubles the Nyquist frequency along the offset-axis.

In another example, high-resolution images may be obtained through SIM or other SR microscopy in connection, e.g., with optimized diffraction-limited imaging. SIM may be implemented by an optical sequencing system to take multiple images of a target object, with varying angles and phase displacements of structured illumination to generate a computational transform (Fourier transform) that is then used to reconstruct closely spaced, otherwise unresolvably high spatial frequency features, into lower frequency signals that may be sensed by an optical system without violating the Abbe diffraction limit. In that manner, captured raw images (e.g., six or nine images) may be assembled into a single image having an extended spatial frequency bandwidth, which may be transformed into real space to generate an image having a higher resolution than one captured by other imaging systems. Other apt SR microscopy systems include, e.g., direct stochastic optical reconstruction microscopy (dSTORM)); photo-activated localization microscopy (PALM)) and stimulated emission depletion microscopy (STED).

In this application, the terms “cluster”, “well”, “sample”, and “fluorescent sample” are interchangeably used because a well contains a corresponding cluster/sample/fluorescent sample. As defined herein, “sample” and its derivatives, is used in its broadest sense and includes any specimen, culture and the like that is suspected of including a target. In some implementations, the sample comprises DNA, RNA, PNA, LNA, chimeric or hybrid forms of nucleic acids. The sample can include any biological, clinical, surgical, agricultural, atmospheric, or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric, or other substrate that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy, or remains of a victim. In some implementations, nucleic acids including one or more target sequences can be obtained from a deceased animal or human. In some implementations, target sequences can include nucleic acids obtained from non-human DNA such a microbial, plant or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target specific primers disclosed herein or one or more target specific primers designed using the primer design criteria outlined herein. In one implementation, a forensic or human identification sample containing at least one target sequence can be amplified using any one or more of the target-specific primers disclosed herein or using the primer criteria outlined herein.

Generally, several implementations will be described herein with respect to a method of analysis. It will be understood that systems are also provided for carrying out the methods in an automated or semi-automated way. Accordingly, this disclosure provides neural network-based template generation and base calling systems, wherein the systems can include a processor; a storage device; and a program for image analysis, the program including instructions for carrying out one or more of the methods set forth herein. Accordingly, the methods set forth herein can be carried out on a computer, for example, having components set forth herein or otherwise known in the art.

The technology disclosed may use neural networks to improve the quality and quantity of nucleic acid sequence information that can be obtained from a nucleic acid sample such as a nucleic acid template or its complement, for instance, a DNA or RNA polynucleotide or other nucleic acid sample. Accordingly, certain implementations of the technology disclosed provide higher throughput polynucleotide sequencing, for instance, higher rates of collection of DNA or RNA sequence data, greater efficiency in sequence data collection, and/or lower costs of obtaining such sequence data, relative to previously available methodologies.

The technology disclosed may neural networks to identify the center of a solid-phase nucleic acid cluster and to analyze optical signals that are generated during sequencing of such clusters, to discriminate unambiguously between adjacent, abutting or overlapping clusters in order to assign a sequencing signal to a single, discrete source cluster. These and related implementations thus permit retrieval of meaningful information, such as sequence data, from regions of high-density cluster arrays where useful information could not previously be obtained from such regions due to confounding effects of overlapping or very closely spaced adjacent clusters, including the effects of overlapping signals (e.g., as used in nucleic acid sequencing) emanating therefrom.

As described in greater detail below, in certain implementations there is provided a composition that comprises a solid support having immobilized thereto one or a plurality of nucleic acid clusters as provided herein. Each cluster comprises a plurality of immobilized nucleic acids of the same sequence and has an identifiable center having a detectable center label as provided herein, by which the identifiable center is distinguishable from immobilized nucleic acids in a surrounding region in the cluster. Also described herein are methods for making and using such clusters that have identifiable centers.

The presently disclosed implementations will find uses in numerous situations where advantages are obtained from the ability to identify, determine, annotate, record or otherwise assign the position of a substantially central location within a cluster, such as high-throughput nucleic acid sequencing, development of image analysis algorithms for assigning optical or other signals to discrete source clusters, and other applications where recognition of the center of an immobilized nucleic acid cluster is desirable and beneficial.

In certain implementations, the present invention contemplates methods that relate to high-throughput nucleic acid analysis such as nucleic acid sequence determination (e.g., “sequencing”). Exemplary high-throughput nucleic acid analyses include without limitation de novo sequencing, re-sequencing, whole genome sequencing, gene expression analysis, gene expression monitoring, epigenetic analysis, genome methylation analysis, allele specific primer extension (APSE), genetic diversity profiling, whole genome polymorphism discovery and analysis, single nucleotide polymorphism analysis, hybridization-based sequence determination methods, and the like. One skilled in the art will appreciate that a variety of different nucleic acids can be analyzed using the solid support AMO nano-constructs the present invention.

Although the implementations of the present invention are described in relation to nucleic acid sequencing, they are applicable in any field where image data acquired at different time points, spatial locations or other temporal or physical perspectives is analyzed. For example, the methods and systems described herein are useful in the fields of molecular and cell biology where image data from microarrays, biological specimens, cells, organisms, and the like are acquired and at different time points or perspectives and analyzed. Images can be obtained using any number of techniques known in the art including, but not limited to, fluorescence microscopy, light microscopy, confocal microscopy, optical imaging, magnetic resonance imaging, tomography scanning and the like. As another example, the methods and systems described herein can be applied where image data obtained by surveillance, aerial or satellite imaging technologies and the like is acquired at different time points or perspectives and analyzed. The methods and systems are particularly useful for analyzing images obtained for a field of view in which the analytes being viewed remain in the same locations relative to each other in the field of view. The analytes may however have characteristics that differ in separate images, for example, the analytes may appear different in separate images of the field of view. For example, the analytes may appear different with regard to the color of a given analyte detected in different images, a change in the intensity of signal detected for a given analyte in different images, or even the appearance of a signal for a given analyte in one image and disappearance of the signal for the analyte in another image.

As used herein, the term “analyte” is intended to mean a point or area in a pattern that can be distinguished from other points or areas according to relative location. An individual analyte can include one or more molecules of a particular type. For example, an analyte can include a single target nucleic acid molecule having a particular sequence or an analyte can include several nucleic acid molecules having the same sequence (and/or complementary sequence, thereof). Different molecules that are at different analytes of a pattern can be differentiated from each other according to the locations of the analytes in the pattern. Example analytes include without limitation, wells in a substrate, beads (or other particles) in or on a substrate, projections from a substrate, ridges on a substrate, pads of gel material on a substrate, or channels in a substrate.

Any of a variety of target analytes that are to be detected, characterized, or identified can be used in an apparatus, system or method set forth herein. Exemplary analytes include, but are not limited to, nucleic acids (e.g., DNA, RNA, or analogs thereof), proteins, polysaccharides, cells, antibodies, epitopes, receptors, ligands, enzymes (e.g., kinases, phosphatases, or polymerases), small molecule drug candidates, cells, viruses, organisms, or the like.

A biopolymeric material herein may include nucleic acid materials, e.g., as analytes, primers, templates, or probes. Nucleic acid materials may be referred to herein as “nucleic acids,” “nucleic acid molecules,” “nucleic acid materials,” “nucleic acid sequences,” “polynucleotides,” or “oligonucleotides,” and can comprise a polymeric form of nucleotides of any length, can comprise DNA and/or RNA, and can be single-stranded, double-stranded, or multiple stranded. One strand of a nucleic acid also refers to its complement. Nucleic acid analytes may be gDNA, including DNA variants (e.g., alleles, polymorphs, missense), mtDNA, mRNA, cDNA transcribed from mRNA, non-coding RNA, and small RNA. Nucleic acid materials herein may also include polynucleotide analogues, amplicons, conjugates, and substitutions, crosslinked polynucleotides, polynucleotide complexes, and non-natural polynucleotides, including, but not limited to, dideoxynucleotides, or biotinylated, aminated, deaminated, alkylated, benzylated, flourophor-labeled polynucleotides. A biopolymeric material herein may also include polypeptides, e.g., as analytes or reagent enzymes. Polypeptide analytes may include functional polypeptides acting, e.g., as effectors, inhibitors, modulators, mediators, transporters, or stimulators in connection with a specific activity affected by a target molecule. Reagent enzymes may include polypeptides involved in nucleic acid synthesis, extension, fragmentation, amplification, or ligation.

In various implementations, nucleic acids may be used as templates as provided herein (e.g., a nucleic acid template, or a nucleic acid complement that is complementary to a nucleic acid nucleic acid template) for particular types of nucleic acid analysis, including but not limited to nucleic acid amplification, nucleic acid expression analysis, and/or nucleic acid sequence determination or suitable combinations thereof. Nucleic acids in certain implementations include, for instance, linear polymers of deoxyribonucleotides in 3′-5′ phosphodiester or other linkages, such as deoxyribonucleic acids (DNA), for example, single- and double-stranded DNA, genomic DNA, copy DNA or complementary DNA (cDNA), recombinant DNA, or any form of synthetic or modified DNA. In other implementations, nucleic acids include for instance, linear polymers of ribonucleotides in 3′-5′ phosphodiester or other linkages such as ribonucleic acids (RNA), for example, single- and double-stranded RNA, messenger (mRNA), copy RNA or complementary RNA (cRNA), alternatively spliced mRNA, ribosomal RNA, small nucleolar RNA (snoRNA), microRNAs (miRNA), small interfering RNAs (sRNA), piwi RNAs (piRNA), or any form of synthetic or modified RNA. Nucleic acids used in the compositions and methods of the present invention may vary in length and may be intact or full-length molecules or fragments or smaller parts of larger nucleic acid molecules. In particular implementations, a nucleic acid may have one or more detectable labels, as described elsewhere herein.

In some implementations, the nucleic acid comprises a plurality of copies of template nucleic acid and/or complements thereof, attached via their 5′ termini to the solid support. Such nucleic acid materials may be referred to “clusters” “colonies,” or “clonal populations.” The copies of nucleic acid strands making up the nucleic acid clusters may be in a single or double stranded form. Copies of a nucleic acid template that are present in a cluster can have nucleotides at corresponding positions that differ from each other, for example, due to presence of a label moiety. The corresponding positions can also contain analog structures having different chemical structure but similar Watson-Crick base-pairing properties, such as is the case for uracil and thymine. Nucleic acid clusters can optionally be created on AMO-based solid supports by amplification, including, e.g., bridge amplification or exclusion amplification (ExAmp) techniques as set forth in further detail elsewhere herein. Multiple repeats of a target sequence can be present in a single nucleic acid molecule, such as a concatemer created using a rolling circle amplification procedure. Such clusters may be characterized by a degree or ratio of monoclonality, or polyclonality.

The nucleic acid clusters of the invention can have different shapes, sizes and densities depending on the conditions used. For example, clusters can have a shape and size that conforms to AMO solid support structures herein. The diameter of a nucleic acid cluster can be designed to be from about 0.2 μm to about 6 μm, about 0.3 μm to about 4 μm, about 0.4 μm to about 3 μm, about 0.5 μm to about 2 μm, about 0.75 μm to about 1.5 μm, or any intervening diameter. In a particular implementation, the diameter of a nucleic acid cluster is about 0.5 μm, about 1 μm, about 1.5 μm, about 2 μm, about 2.5 μm, about 3 μm, about 4 μm, about 5 μm, or about 6 μm. The diameter of a nucleic acid cluster may be influenced by a number of parameters, including, but not limited to the number of amplification cycles performed in producing the cluster, the length of the nucleic acid template or the density of primers attached to the surface upon which clusters are formed. The density of nucleic acid clusters can be designed to typically be in the range of 0.1/mm², 1/mm², 10/mm², 100/mm², 1,000/mm², 10,000/mm² to 100,000/mm². The present invention further contemplates, in part, higher density nucleic acid clusters, for example, 100,000/mm² to 1,000,000/mm² and 1,000,000/mm² to 10,000,000/mm².

As used herein, an “analyte” is an area of interest within a specimen or field of view. When used in connection with microarray devices or other molecular analytical devices, an analyte refers to the area occupied by similar or identical molecules. For example, an analyte can be an amplified oligonucleotide or any other group of a polynucleotide or polypeptide with a same or similar sequence. In other implementations, an analyte can be any element or group of elements that occupy a physical area on a specimen. For example, an analyte could be a parcel of land, a body of water or the like. When an analyte is imaged, each analyte will have some area. Thus, in many implementations, an analyte is not merely one pixel.

The distances between analytes can be described in any number of ways. In some implementations, the distances between analytes can be described from the center of one analyte to the center of another analyte. In other implementations, the distances can be described from the edge of one analyte to the edge of another analyte, or between the outer-most identifiable points of each analyte. The edge of an analyte can be described as the theoretical or actual physical boundary on a chip, or some point inside the boundary of the analyte. In other implementations, the distances can be described in relation to a fixed point on the specimen or in the image of the specimen.

The size of an analyte on an array (or other object used in a method or system herein) can be selected to suit a particular application. For example, in some implementations, an analyte of an array can have a size that accommodates only a single nucleic acid molecule. A surface having a plurality of analytes in this size range is useful for constructing an array of molecules for detection at single molecule resolution. Analytes in this size range are also useful for use in arrays having analytes that each contain a colony of nucleic acid molecules. Thus, the analytes of an array can each have an area that is no larger than about 1 mm², no larger than about 500 μm², no larger than about 100 μm², no larger than about 10 μm², no larger than about 1 μm², no larger than about 500 nm², or no larger than about 100 nm², no larger than about 10 nm², no larger than about 5 nm², or no larger than about 1 nm². Alternatively or additionally, the analytes of an array will be no smaller than about 1 mm², no smaller than about 500 μm², no smaller than about 100 μm², no smaller than about 10 μm², no smaller than about 1 μm², no smaller than about 500 nm², no smaller than about 100 nm², no smaller than about 10 nm², no smaller than about 5 nm², or no smaller than about 1 nm². Indeed, an analyte can have a size that is in a range between an upper and lower limit selected from those exemplified above. Although several size ranges for analytes of a surface have been exemplified with respect to nucleic acids and on the scale of nucleic acids, it will be understood that analytes in these size ranges can be used for applications that do not include nucleic acids. It will be further understood that the size of the analytes need not necessarily be confined to a scale used for nucleic acid applications.

For implementations that include an object having a plurality of analytes, such as an array of analytes, the analytes can be discrete, being separated with spaces between each other. An array useful in the invention can have analytes that are separated by edge to edge distance of at most 100 μm, 50 μm, 10 μm, 5 μm, 1 μm, 0.5 μm, or less. Alternatively or additionally, an array can have analytes that are separated by an edge to edge distance of at least 0.5 μm, 1 μm, 5 μm, 10 μ

The average pitch in a regular pattern can be at most 100 μm, 50 μm, 10 μm, 5 μm, 1 μm, 0.5 μm, or less. Alternatively or additionally, the average pitch in a regular pattern can be at least 0.5 μm, 1 μm, 5 μm, 10 μm, 50 μm, 100 μm, or more. These ranges can apply to the maximum or minimum pitch for a regular pattern as well. For example, the maximum analyte pitch for a regular pattern can be at most 100 μm, 50 μm, 10 μm, 5 μm, 1 μm, 0.5 μm, or less; and/or the minimum analyte pitch in a regular pattern can be at least 0.5 μm, 1 μm, 5 μm, 10 μm, 50 μm, 100 μm, or more.

The density of analytes in an array can also be understood in terms of the number of analytes present per unit area. For example, the average density of analytes for an array can be at least about 1×10³ analytes/mm², 1×10⁴ analytes/mm², 1×10⁵ analytes/mm², 1×10⁶ analytes/mm², 1×10⁷ analytes/mm², 1×10⁸ analytes/mm², or 1×10⁹ analytes/mm², or higher. Alternatively or additionally the average density of analytes for an array can be at most about 1×10⁹ analytes/mm², 1×10⁸ analytes/mm², 1×10⁷ analytes/mm², 1×10⁶ analytes/mm², 1×10⁵ analytes/mm², 1×10⁴ analytes/mm², or 1×10³ analytes/mm², or less.

The size and shape of analytes in a pattern may be determined by the size and shape of nanostructures in an array. For example, when observed in a two-dimensional plane, such as on the surface of an array, the analytes can appear rounded, circular, oval, rectangular, square, symmetric, asymmetric, triangular, polygonal, or the like. The analytes can be arranged in a regular repeating pattern including, for example, a hexagonal or rectilinear pattern. A pattern can be selected to achieve a desired level of packing. For example, round analytes are optimally packed in a hexagonal arrangement. Of course, other packing arrangements can also be used for round analytes and vice versa.

A pattern can be characterized in terms of the number of analytes that are present in a subset that forms the smallest geometric unit of the pattern. The subset can include, for example, at least about 2, 3, 4, 5, 6, 10 or more analytes. Depending upon the size and density of the analytes the geometric unit can occupy an area of less than 1 mm², 500 μm², 100 μm², 50 μm², 10 μm², 1 μm², 500 nm², 100 nm², 50 nm², 10 nm², or less. Alternatively or additionally, the geometric unit can occupy an area of greater than 10 nm², 50 nm², 100 nm², 500 nm², 1 μm², 10 μm², 50 μm², 100 μm², 500 μm², 1 mm², or more. Characteristics of the analytes in a geometric unit, such as shape, size, pitch, and the like, can be selected from those set forth herein more generally with regard to analytes in an array or pattern.

An array having a regular pattern of analytes can be ordered with respect to the relative locations of the analytes but random with respect to one or more other characteristic of each analyte. For example, in the case of a nucleic acid array, the nuclei acid analytes can be ordered with respect to their relative locations but random with respect to one's knowledge of the sequence for the nucleic acid species present at any particular analyte. As a more specific example, nucleic acid arrays formed by seeding a repeating pattern of analytes with template nucleic acids and amplifying the template at each analyte to form copies of the template at the analyte (e.g., via cluster amplification or bridge amplification) will have a regular pattern of nucleic acid analytes but will be random with regard to the distribution of sequences of the nucleic acids across the array. Thus, detection of the presence of nucleic acid material generally on the array can yield a repeating pattern of analytes, whereas sequence specific detection can yield non-repeating distribution of signals across the array.

As used herein, the term “flow cell” is intended to mean a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis, and is capable of releasing the surface chemistry either electrochemically or upon exposure to visible light. The flow cell also includes an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a patterned or nonpatterned structure and a lid. In other examples, the flow channel may be defined between two patterned or non-patterned structures that are bonded together.

The term “solid support” refers to a structure upon which various chemistry (e.g., polymeric hydrogel, primers, etc.) may be added in connection with molecular analysis contemplated herein. The substrate may be a wafer, a panel, a rectangular sheet, a die, or any other suitable configuration. The substrate is generally rigid and is insoluble in an aqueous liquid. The substrate may be a single layer structure, or a multi-layered structure (e.g., including a support and a patterned material on the support). Examples of suitable substrates will be described further herein.

For convenience, various example embodiments described herein include methods and compositions for flow-cell based sequencing, e.g., of clonal populations of a nucleic acid library clustered on an array of bead substrates; orthogonal reagents and complimentary chemistry for functionalization of a flow cell surface for selective capture, in situ enrichment, imaging, and traceless release of a nucleic acid sample in a sequencing cycle. AMO-based solid support nanoconstructs herein may be supported on a variety of architectures, for a variety of molecular analyses, within a variety of analytic, implemented in variety of systems or platforms, for a variety of genomic, transcriptomic, proteomic, and applications-particularly in contexts in which a biological sample is presented in a repeating pattern of analytes in an x-y plane.

For example, AMO-based solid support nanoconstructs and patterned substrates may be implemented on microarrays useful, e.g., in connection with genotyping assays, systems and platforms. Microarrays typically include deoxyribonucleic acid (DNA) or ribonucleic acid (RNA) probes. These are specific for nucleotide sequences present in humans and other organisms. In certain applications, for example, individual DNA or RNA probes can be attached at individual analytes of an array. A test sample, such as from a known person or organism, can be exposed to the array, such that target nucleic acids (e.g., gene fragments, mRNA, or amplicons thereof) hybridize to complementary probes at respective analytes in the array. The probes can be labeled in a target specific process (e.g., due to labels present on the target nucleic acids or due to enzymatic labeling of the probes or targets that are present in hybridized form at the analytes). The array can then be examined by scanning specific frequencies of light over the analytes to identify which target nucleic acids are present in the sample.

Biological microarrays may be used for genetic sequencing and similar applications. In general, genetic sequencing comprises determining the order of nucleotides in a length of target nucleic acid, such as a fragment of DNA or RNA. Relatively short sequences are typically sequenced at each analyte, and the resulting sequence information may be used in various bioinformatics methods to logically fit the sequence fragments together so as to reliably determine the sequence of much more extensive lengths of genetic material from which the fragments were derived. Automated, computer-based algorithms for characteristic fragments have been developed, and have been used more recently in genome mapping, identification of genes and their function, and so forth. Microarrays are particularly useful for characterizing genomic content because a large number of variants are present and this supplants the alternative of performing many experiments on individual probes and targets. The microarray is an ideal format for performing such investigations in a practical manner.

Any of a variety of microarrays may be used in a method or system set forth herein. A typical array contains analytes, each having an individual probe or a population of probes. In the latter case, the population of probes at each analyte is typically homogenous having a single species of probe. For example, in the case of a nucleic acid array, each analyte can have multiple nucleic acid molecules each having a common sequence. However, in some implementations the populations at each analyte of an array can be heterogeneous. Similarly, protein arrays can have analytes with a single protein or a population of proteins typically, but not always, having the same amino acid sequence. The probes can be attached to the surface of an array for example, via covalent linkage of the probes to the surface or via non-covalent interaction(s) of the probes with the surface. In some implementations, probes, such as nucleic acid molecules, can be attached to a surface via a gel layer as described, for example, in U.S. patent application Ser. No. 13/784,368 and U.S. Pat. App. Pub. No. 2011/0059865 A1, each of which is incorporated herein by reference.

Example arrays include, without limitation, a BeadChip Array available from Illumina, Inc. (San Diego, Calif.) or others such as those where probes are attached to beads that are present on a surface (e.g., beads in wells on a surface) such as those described in U.S. Pat. Nos. 6,266,459; 6,355,431; 6,770,441; 6,859,570; or 7,622,294; or PCT Publication No. WO 00/63437, each of which is incorporated herein by reference. Further examples of commercially available microarrays that can be used include, for example, an Affymetrix® GeneChip® microarray or other microarray synthesized in accordance with techniques sometimes referred to as VLSIPS™ (Very Large Scale Immobilized Polymer Synthesis) technologies. A spotted microarray can also be used in a method or system according to some implementations of the present disclosure. An example spotted microarray is a CodeLink™ Array available from Amersham Biosciences. Another microarray that is useful is one that is manufactured using inkjet printing methods such as SurePrint™ Technology available from Agilent Technologies.

Other useful arrays include those that are used in nucleic acid sequencing applications. For example, arrays having amplicons of genomic fragments (often referred to as clusters) are particularly useful such as those described in Bentley et al., Nature 456:53-59 (2008), WO 4/018497; WO 91/06678; WO 7/123744; U.S. Pat. Nos. 7,329,492; 7,211,414; 7,315,19; 7,405,281, or 7,057,26; or U.S. Pat. App. Pub. No. 2008/0108082 A1, each of which is incorporated herein by reference. Another type of array that is useful for nucleic acid sequencing is an array of particles produced from an emulsion PCR technique. Examples are described in Dressman et al., Proc. Natl. Acad. Sci. USA 100:8817-8822 (2003), WO 5/010145, U.S. Pat. App. Pub. No. 2005/0130173, or U.S. Pat. App. Pub. No. 2005/0064460, each of which is incorporated herein by reference in its entirety.

Patterned arrays herein can be used for nucleic acid sequencing or other analytical applications. Example patterned arrays, methods for their manufacture and methods for their use are set forth in U.S. Ser. No. 13/787,396; U.S. Ser. No. 13/783,43; U.S. Ser. No. 13/784,368; U.S. Pat. App. Pub. No. 2013/0116153 A1; and U.S. Pat. App. Pub. No. 2012/0316086 A1, each of which is incorporated herein by reference. The analytes of such patterned arrays can be used to capture a single nucleic acid template molecule to seed subsequent formation of a monoclonal population, for example, via bridge amplification or ExAmp. Such patterned arrays are particularly useful for nucleic acid sequencing applications.

In one embodiment, patterned AMO films herein may be adapted for use as a passivation layer with CMOS sensor technology. For example, an AMO film for use as a blanket layer for CMOS passivation may be patterned on an underside surface to align with underlying CMOS features.

As used herein, the term “template” refers to a representation of the location or relation between signals or analytes. Thus, in some implementations, a template is a physical grid with a representation of signals corresponding to analytes in a specimen. In some implementations, a template can be a chart, table, text file or other computer file indicative of locations corresponding to analytes. In implementations presented herein, a template is generated to track the location of analytes of a specimen across a set of images of the specimen captured at different reference points. For example, a template could be a set of x, y coordinates or a set of values that describe the direction and/or distance of one analyte with respect to another analyte.

A base refers to a nucleotide base or nucleotide, A (adenine), C (cytosine), T (thymine), or G (guanine). This application uses “base(s)” and “nucleotide(s)” interchangeably.

The term “chromosome” refers to the heredity-bearing gene carrier of a living cell, which is derived from chromatin strands comprising DNA and protein components (especially histones). The conventional internationally recognized individual human genome chromosome numbering system is employed herein.

The term “site” refers to a unique position (e.g., chromosome ID, chromosome position and orientation) on a reference genome. In some implementations, a site may be a residue, a sequence tag, or a segment's position on a sequence. The term “locus” may be used to refer to the specific location of a nucleic acid sequence or polymorphism on a reference chromosome.

The term “sample” herein refers to a sample, typically derived from a biological fluid, cell, tissue, organ, or organism containing a nucleic acid or a mixture of nucleic acids containing at least one nucleic acid sequence that is to be sequenced and/or phased. Such samples include, but are not limited to sputum/oral fluid, amniotic fluid, blood, a blood fraction, fine needle biopsy samples (e.g., surgical biopsy, fine needle biopsy, etc.), urine, peritoneal fluid, pleural fluid, tissue explant, organ culture and any other tissue or cell preparation, or fraction or derivative thereof or isolated therefrom. Although the sample is often taken from a human subject (e.g., patient), samples can be taken from any organism having chromosomes, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc.

The term “sequence” includes or represents a strand of nucleotides coupled to each other. The nucleotides may be based on DNA or RNA. It should be understood that one sequence may include multiple sub-sequences. For example, a single sequence (e.g., of a PCR amplicon) may have 350 nucleotides. The sample read may include multiple sub-sequences within these 350 nucleotides. For instance, the sample read may include first and second flanking subsequences having, for example, 20-50 nucleotides. The first and second flanking sub-sequences may be located on either side of a repetitive segment having a corresponding sub-sequence (e.g., 40-100 nucleotides). Each of the flanking sub-sequences may include (or include portions of) a primer sub-sequence (e.g., 10-30 nucleotides). For ease of reading, the term “sub-sequence” will be referred to as “sequence,” but it is understood that two sequences are not necessarily separate from each other on a common strand. To differentiate the various sequences described herein, the sequences may be given different labels (e.g., target sequence, primer sequence, flanking sequence, reference sequence, and the like). Other terms, such as “allele,” may be given different labels to differentiate between like objects. The application uses “read(s)” and “sequence read(s)” interchangeably.

The term “paired-end sequencing” refers to sequencing methods that sequence both ends of a target fragment. Paired-end sequencing may facilitate detection of genomic rearrangements and repetitive segments, as well as gene fusions and novel transcripts. Methodology for paired-end sequencing is described in PCT publication WO07010252, PCT application Serial No. PCTGB2007/003798 and U.S. patent application publication U.S. 2009/0088327, each of which is incorporated by reference herein. In one example, a series of operations may be performed as follows; (a) generate clusters of nucleic acids; (b) linearize the nucleic acids; (c) hybridize a first sequencing primer and carry out repeated cycles of extension, scanning and deblocking, as set forth above; (d) “invert” the target nucleic acids on the flow cell surface by synthesizing a complimentary copy; (c) linearize the resynthesized strand; and (f) hybridize a second sequencing primer and carry out repeated cycles of extension, scanning and deblocking, as set forth above. The inversion operation can be carried out be delivering reagents as set forth above for a single cycle of bridge amplification.

The term “reference genome” or “reference sequence” refers to any particular known genome sequence, whether partial or complete, of any organism which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences. A genome includes both the genes and the noncoding sequences of the DNA. The reference sequence may be larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference genome sequence is that of a full-length human genome. In another example, the reference genome sequence is limited to a specific human chromosome such as chromosome 13. In some implementations, a reference chromosome is a chromosome sequence from human genome version hg19. Such sequences may be referred to as chromosome reference sequences, although the term reference genome is intended to cover such sequences. Other examples of reference sequences include genomes of other species, as well as chromosomes, sub-chromosomal regions (such as strands), etc., of any species. In various implementations, the reference genome is a consensus sequence or other combination derived from multiple individuals. However, in certain applications, the reference sequence may be taken from a particular individual. In other implementations, the “genome” also covers so-called “graph genomes”, which use a particular storage format and representation of the genome sequence. In one implementation, graph genomes store data in a linear file. In another implementation, the graph genomes refer to a representation where alternative sequences (e.g., different copies of a chromosome with small differences) are stored as different paths in a graph. Additional information regarding graph genome implementations can be found in https://www.biorxiv.org/content/biorxiv/early/2018/3/20/194530.full.pdf, the content of which is hereby incorporated herein by reference in its entirety.

The term “read” refer to a collection of sequence data that describes a fragment of a nucleotide sample or reference. The term “read” may refer to a sample read and/or a reference read. Typically, though not necessarily, a read represents a short sequence of contiguous base pairs in the sample or reference. The read may be represented symbolically by the base pair sequence (in ATCG) of the sample or reference fragment. It may be stored in a memory device and processed as appropriate to determine whether the read matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

Next-generation sequencing methods include, for example, sequencing by synthesis technology (Illumina), pyrosequencing (454), ion semiconductor technology (Ion Torrent sequencing), single-molecule real-time sequencing (Pacific Biosciences) and sequencing by ligation (SOLID sequencing). Depending on the sequencing methods, the length of each read may vary from about 30 bp to more than 10,000 bp. For example, the DNA sequencing method using SOLID sequencer generates nucleic acid reads of about 50 bp. For another example, Ion Torrent Sequencing generates nucleic acid reads of up to 400 bp and 454 pyrosequencing generates nucleic acid reads of about 700 bp. For yet another example, single-molecule real-time sequencing methods may generate reads of 10,000 bp to 15,000 bp. Therefore, in certain implementations, the nucleic acid sequence reads have a length of 30-100 bp, 50-200 bp, or 50-400 bp.

The terms “sample read”, “sample sequence” or “sample fragment” refer to sequence data for a genomic sequence of interest from a sample. For example, the sample read comprises sequence data from a PCR amplicon having a forward and reverse primer sequence. The sequence data can be obtained from any select sequence methodology. The sample read can be, for example, from a sequencing-by-synthesis (SBS) reaction, a sequencing-by-ligation reaction, or any other suitable sequencing methodology for which it is desired to determine the length and/or identity of a repetitive element. The sample read can be a consensus (e.g., averaged or weighted) sequence derived from multiple sample reads. In certain implementations, providing a reference sequence comprises identifying a locus-of-interest based upon the primer sequence of the PCR amplicon.

The term “raw fragment” refers to sequence data for a portion of a genomic sequence of interest that at least partially overlaps a designated position or secondary position of interest within a sample read or sample fragment. Non-limiting examples of raw fragments include a duplex stitched fragment, a simplex stitched fragment, a duplex un-stitched fragment, and a simplex un-stitched fragment. The term “raw” is used to indicate that the raw fragment includes sequence data having some relation to the sequence data in a sample read, regardless of whether the raw fragment exhibits a supporting variant that corresponds to and authenticates or confirms a potential variant in a sample read. The term “raw fragment” does not indicate that the fragment necessarily includes a supporting variant that validates a variant call in a sample read. For example, when a sample read is determined by a variant call application to exhibit a first variant, the variant call application may determine that one or more raw fragments lack a corresponding type of “supporting” variant that may otherwise be expected to occur given the variant in the sample read.

The terms “mapping”, “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain implementations, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

The term “indel” refers to the insertion and/or the deletion of bases in the DNA of an organism. A micro-indel represents an indel that results in a net change of 1 to 50 nucleotides. In coding regions of the genome, unless the length of an indel is a multiple of 3, it will produce a frameshift mutation. Indels can be contrasted with point mutations. An indel inserts and deletes nucleotides from a sequence, while a point mutation is a form of substitution that replaces one of the nucleotides without changing the overall number in the DNA. Indels can also be contrasted with a Tandem Base Mutation (TBM), which may be defined as substitution at adjacent nucleotides (primarily substitutions at two adjacent nucleotides, but substitutions at three adjacent nucleotides have been observed.

The term “variant” refers to a nucleic acid sequence that is different from a nucleic acid reference. Typical nucleic acid sequence variant includes without limitation single nucleotide polymorphism (SNP), short deletion and insertion polymorphisms (Indel), copy number variation (CNV), microsatellite markers or short tandem repeats and structural variation. Somatic variant calling is the effort to identify variants present at low frequency in the DNA sample. Somatic variant calling is of interest in the context of cancer treatment. Cancer is caused by an accumulation of mutations in DNA. A DNA sample from a tumor is generally heterogeneous, including some normal cells, some cells at an early stage of cancer progression (with fewer mutations), and some late-stage cells (with more mutations). Because of this heterogeneity, when sequencing a tumor (e.g., from an FFPE sample), somatic mutations will often appear at a low frequency. For example, a SNV might be seen in only 10% of the reads covering a given base. A variant that is to be classified as somatic or germline by the variant classifier is also referred to herein as the “variant under test”.

The term “noise” refers to a mistaken variant call resulting from one or more errors in the sequencing process and/or in the variant call application.

The term “variant frequency” represents the relative frequency of an allele (variant of a gene) at a particular locus in a population, expressed as a fraction or percentage. For example, the fraction or percentage may be the fraction of all chromosomes in the population that carry that allele. By way of example, sample variant frequency represents the relative frequency of an allele/variant at a particular locus/position along a genomic sequence of interest over a “population” corresponding to the number of reads and/or samples obtained for the genomic sequence of interest from an individual. As another example, a baseline variant frequency represents the relative frequency of an allele/variant at a particular locus/position along one or more baseline genomic sequences where the “population” corresponding to the number of reads and/or samples obtained for the one or more baseline genomic sequences from a population of normal individuals.

The term “variant allele frequency (VAF)” refers to the percentage of sequenced reads observed matching the variant divided by the overall coverage at the target position. VAF is a measure of the proportion of sequenced reads carrying the variant.

The terms “position”, “designated position”, and “locus” refer to a location or coordinate of one or more nucleotides within a sequence of nucleotides. The terms “position”, “designated position”, and “locus” also refer to a location or coordinate of one or more base pairs in a sequence of nucleotides.

The term “haplotype” refers to a combination of alleles at adjacent sites on a chromosome that are inherited together. A haplotype may be one locus, several loci, or an entire chromosome depending on the number of recombination events that have occurred between a given set of loci, if any occurred.

The term “threshold” herein refers to a numeric or non-numeric value that is used as a cutoff to characterize a sample, a nucleic acid, or portion thereof (e.g., a read). A threshold may be varied based upon empirical analysis. The threshold may be compared to a measured or calculated value to determine whether the source giving rise to such value suggests should be classified in a particular manner. Threshold values can be identified empirically or analytically. The choice of a threshold is dependent on the level of confidence that the user wishes to have to make the classification. The threshold may be chosen for a particular purpose (e.g., to balance sensitivity and selectivity). As used herein, the term “threshold” indicates a point at which a course of analysis may be changed and/or a point at which an action may be triggered. A threshold is not required to be a predetermined number. Instead, the threshold may be, for instance, a function that is based on a plurality of factors. The threshold may be adaptive to the circumstances. Moreover, a threshold may indicate an upper limit, a lower limit, or a range between limits.

In some implementations, a metric or score that is based on sequencing data may be compared to the threshold. As used herein, the terms “metric” or “score” may include values or results that were determined from the sequencing data or may include functions that are based on the values or results that were determined from the sequencing data. Like a threshold, the metric or score may be adaptive to the circumstances. For instance, the metric or score may be a normalized value. As an example of a score or metric, one or more implementations may use count scores when analyzing the data. A count score may be based on number of sample reads. The sample reads may have undergone one or more filtering stages such that the sample reads have at least one common characteristic or quality. For example, each of the sample reads that are used to determine a count score may have been aligned with a reference sequence or may be assigned as a potential allele. The number of sample reads having a common characteristic may be counted to determine a read count. Count scores may be based on the read count. In some implementations, the count score may be a value that is equal to the read count. In other implementations, the count score may be based on the read count and other information. For example, a count score may be based on the read count for a particular allele of a genetic locus and a total number of reads for the genetic locus. In some implementations, the count score may be based on the read count and previously-obtained data for the genetic locus. In some implementations, the count scores may be normalized scores between predetermined values. The count score may also be a function of read counts from other loci of a sample or a function of read counts from other samples that were concurrently run with the sample-of-interest. For instance, the count score may be a function of the read count of a particular allele and the read counts of other loci in the sample and/or the read counts from other samples. As one example, the read counts from other loci and/or the read counts from other samples may be used to normalize the count score for the particular allele.

The terms “coverage” or “fragment coverage” refer to a count or other measure of a number of sample reads for the same fragment of a sequence. A read count may represent a count of the number of reads that cover a corresponding fragment. Alternatively, the coverage may be determined by multiplying the read count by a designated factor that is based on historical knowledge, knowledge of the sample, knowledge of the locus, etc.

The term “read depth” (conventionally a number followed by “x”) refers to the number of sequenced reads with overlapping alignment at the target position. This is often expressed as an average or percentage exceeding a cutoff over a set of intervals (such as exons, genes, or panels). For example, a clinical report might say that a panel average coverage is 1,105× with 98% of targeted bases covered >100×.

The terms “base call quality score” or “Q score” refer to a PHRED-scaled probability ranging from 0-50 inversely proportional to the probability that a single sequenced base is correct. For example, a T base call with Q of 20 is considered likely correct with a probability of 99.99%. Any base call with Q<20 should be considered low quality, and any variant identified where a substantial proportion of sequenced reads supporting the variant are of low quality should be considered potentially false positive.

The terms “variant reads” or “variant read number” refer to the number of sequenced reads supporting the presence of the variant.

The reads alignment (also called reads mapping) is the process of figuring out where in the genome a sequence is from. Once the alignment is performed, the “mapping quality” or the “mapping quality score (MAPQ)” of a given read quantifies the probability that its position on the genome is correct. The mapping quality is encoded in the phred scale where P is the probability that the alignment is not correct. The probability is calculated as: P=10^{(-MAQ/10)}, where MAPQ is the mapping quality. For example, a mapping quality of 40=10 to the power of −4, meaning that there is a 0.01% chance that the read was aligned incorrectly. The mapping quality is therefore associated with several alignment factors, such as the base quality of the read, the complexity of the reference genome, and the paired-end information. Regarding the first, if the base quality of the read is low, it means that the observed sequence might be wrong and thus its alignment is wrong. Regarding the second, the mappability refers to the complexity of the genome. Repeated regions are more difficult to map and reads falling in these regions usually get low mapping quality. In this context, the MAPQ reflects the fact that the reads are not uniquely aligned and that their real origin cannot be determined. Regarding the third, in case of paired-end sequencing data, concordant pairs are more likely to be well aligned. The higher the mapping quality, the better the alignment. A read aligned with a good mapping quality usually means that the read sequence was good and was aligned with few mismatches in a high mappability region. The MAPQ value can be used as a quality control of the alignment results. The proportion of reads aligned with an MAPQ higher than 20 is usually for downstream analysis.

As used herein, a “signal” refers to a detectable event such as an emission, such as light emission, for example, in an image. Thus, in some implementations, a signal can represent any detectable light emission that is captured in an image (i.e., a “spot”). Thus, as used herein, “signal” can refer to both an actual emission from an analyte of the specimen, and can refer to a spurious emission that does not correlate to an actual analyte. Thus, a signal could arise from noise and could be later discarded as not representative of an actual analyte of a specimen.

As used herein, “crosstalk” refers to the detection of signals in one image that are also detected in a separate image. In some implementations, crosstalk can occur when an emitted signal is detected in two separate detection channels. For example, where an emitted signal occurs in one color, the emission spectrum of that signal may overlap with another emitted signal in another color. In some implementations, fluorescent molecules used to indicate the presence of nucleotide bases A, C, G and T are detected in separate channels. However, because the emission spectra of A and C overlap, some of the C color signal may be detected during detection using the A color channel. Accordingly, crosstalk between the A and C signals allows signals from one color image to appear in the other color image. In some implementations, G and T crosstalk. In some implementations, the amount of crosstalk between channels is asymmetric. It will be appreciated that the amount of crosstalk between channels can be controlled by, among other things, the selection of signal molecules having an appropriate emission spectrum as well as selection of the size and wavelength range of the detection channel.

As used herein, the term “fiducial” is intended to mean a distinguishable point of reference in or on an object. The point of reference can be, for example, a mark, second object, shape, edge, area, irregularity, channel, pit, post, or the like. The point of reference can be present in an image of the object or in another data set derived from detecting the object. The point of reference can be specified by an x and/or y coordinate in a plane of the object. Alternatively or additionally, the point of reference can be specified by a z coordinate that is orthogonal to the xy plane, for example, being defined by the relative locations of the object and a detector. One or more coordinates for a point of reference can be specified relative to one or more other analytes of an object or of an image or other data set derived from the object.

As used herein, the term “optical signal” is intended to include, for example, fluorescent, luminescent, scatter, or absorption signals. Optical signals can be detected in the ultraviolet (UV) range (about 200 to 390 nm), visible (VIS) range (about 391 to 770 nm), infrared (IR) range (about 0.771 to 25 microns), or other range of the electromagnetic spectrum. Optical signals can be detected in a way that excludes all or part of one or more of these ranges.

As used herein, the term “signal level” is intended to mean an amount or quantity of detected energy or coded information that has a desired or predefined characteristic. For example, an optical signal can be quantified by one or more of intensity, wavelength, energy, frequency, power, luminance, or the like. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

As used herein, the term “xy coordinates” is intended to mean information that specifies location, size, shape, and/or orientation in an xy plane. The information can be, for example, numerical coordinates in a Cartesian system. The coordinates can be provided relative to one or both of the x and y axes or can be provided relative to another location in the xy plane. For example, coordinates of an analyte of an object can specify the location of the analyte relative to location of a fiducial or other analyte of the object.

As used herein, the term “xy plane” is intended to mean a 2-dimensional area defined by straight line axes x and y. When used in reference to a detector and an object observed by the detector, the area can be further specified as being orthogonal to the direction of observation between the detector and object being detected.

As used herein, the term “z coordinate” is intended to mean information that specifies the location of a point, line or area along an axis that is orthogonal to an xy plane. In particular implementations, the z axis is orthogonal to an area of an object that is observed by a detector. For example, the direction of focus for an optical system may be specified along the z axis.

In some implementations, acquired signal data is transformed using an affine transformation. In some such implementations, template generation makes use of the fact that the affine transforms between color channels are consistent between runs. Because of this consistency, a set of default offsets can be used when determining the coordinates of the analytes in a specimen. For example, a default offsets file can contain the relative transformation (shift, scale, skew) for the different channels relative to one channel, such as the A channel. In other implementations, however, the offsets between color channels drift during a run and/or between runs, making offset-driven template generation difficult. In such implementations, the methods and systems provided herein can utilize offset-less template generation, which is described further below.

In some of the systems for image analysis described herein, each image in the set of images includes color signals, wherein a different color corresponds to a different nucleotide base. In some aspects, each image of the set of images comprises signals having a single color selected from at least four different colors. In some aspects, each image in the set of images comprises signals having a single color selected from four different colors. In some of the systems described herein, nucleic acids can be sequenced by providing four different labeled nucleotide bases to the array of molecules so as to produce four different images, each image comprising signals having a single color, wherein the signal color is different for each of the four different images, thereby producing a cycle of four-color images that corresponds to the four possible nucleotides present at a particular position in the nucleic acid. In certain aspects, the system comprises a flow cell that is configured to deliver additional labeled nucleotide bases to the array of molecules, thereby producing a plurality of cycles of color images.

In some implementations, the methods provided herein can include determining whether a processor is actively acquiring data or whether the processor is in a low activity state. Acquiring and storing large numbers of high-quality images typically requires massive amounts of storage capacity. Additionally, once acquired and stored, the analysis of image data can become resource intensive and can interfere with processing capacity of other functions, such as ongoing acquisition and storage of additional image data. Accordingly, as used herein, the term low activity state refers to the processing capacity of a processor at a given time. In some implementations, a low activity state occurs when a processor is not acquiring and/or storing data. In some implementations, a low activity state occurs when some data acquisition and/or storage is taking place, but additional processing capacity remains such that image analysis can occur at the same time without interfering with other functions.

Also provided herein are systems for performing image analysis. The systems can include a processor; a storage capacity; and a program for image analysis, the program comprising instructions for processing a first data set for storage and the second data set for analysis, wherein the processing comprises acquiring and/or storing the first data set on the storage device and analyzing the second data set when the processor is not acquiring the first data set. In certain aspects, the program includes instructions for identifying at least one instance of a conflict between acquiring and/or storing the first data set and analyzing the second data set; and resolving the conflict in favor of acquiring and/or storing image data such that acquiring and/or storing the first data set is given priority. In certain aspects, the first data set comprises image files obtained from an optical imaging device. In certain aspects, the system further comprises an optical imaging device. In some aspects, the optical imaging device comprises a light source and a detection device.

### Example AMO Supported Flow Cell Architecture

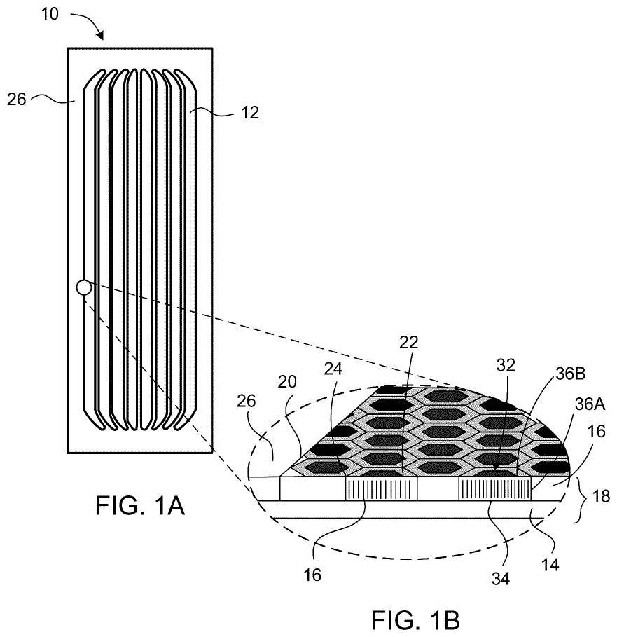

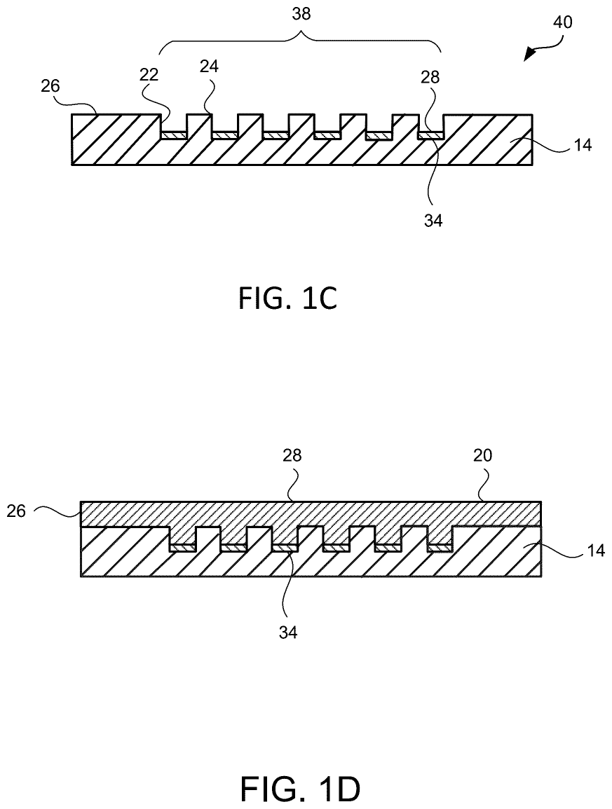

One example of AMO supported flow cell architecture is the flow cell 10 as shown in FIGS. 1A and 1B. Generally, flow cell 10 may include a patterned structure, e.g., an array of depressions 32, as shown in FIG. 1B, and the patterned structure may be organized into lanes, each separated by non-patterned, non-functionalized interstitial regions, which may be bonded to a lid 20 to form flow channels 12 along each lane of patterned structure. The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). Each flow channel 12 may be isolated from another flow channel 12 so that fluid introduced into a flow channel 12 does not flow into adjacent flow channel(s) 12. Some examples of the fluids introduced into the flow channel 12 may introduce sample and reaction components (for NGS: e.g., DNA sample, polymerases, sequencing primers, nucleotides, etc.), washing solutions, deblocking agents, etc.

As illustrated in FIG. 1B, the flow channel 12 may include a multi-layered or composite structure 18, which includes, a minimum, a single layer base support 14 overlayed with an AMO film layer 16.

The support layer may be any suitable low-background material, including materials exhibiting both high transmissivity and high fluorescence transparency, particularly for use as solid supports for fluorescence-based imaging implementations. Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, silica-based materials, aluminum silicate, silicon and modified silicon (e.g., boron doped p+ silicon), silicon nitride (Si3N4), silicon oxide (SiO2), tantalum pentoxide (Ta2O5) or other tantalum oxide(s) (TaOx), hafnium oxide (HfO2), carbon, metals, inorganic glasses, or the like. In one example, single layer base supports 14 is a glass, for example, alkaline earth boro-aluminosilicate glass (e.g., EAGLE XG® (Corning, NY)), which has an annealing point (10¹³ poises) rated ˜1332° F.

The AMO film layer 16 may have thickness sufficient to accommodate the depth of depression 32, which can range, for example, between 0.01 nm to 450 nm depending on the application. As discussed in greater detail here, in one example (shown in FIG. 1B), the thickness of the AMO film layer may be coterminous with the depth of depression 32, such that a base portion of the inner surface of depression 32 exposes a surface of the single layer base supports 14. Alternatively, the thickness of the AMO film layer may accommodate the entire depth of depression 32, such the entire inner surface of depression 32 is formed in the material of the AMO film layer.

The AMO film may be processed from any suitable metal oxide (including dioxides and trioxides) that forms natural porous structures under anodic oxidation as described herein, including, e.g., aluminum oxide anodized to anodic aluminum oxide (AAO), and titanium oxide anodized to anodic titanium oxide (ATiO), hafnium oxide (HfO2) anodized to anodic hafnium oxide (AHO), tantalum oxide anodized to anodic tantalum oxide (ATaO), niobium oxide anodized to anodic niobium oxide (ANO), cerium oxide anodized to anodic cerium oxide (ACO), and gallium oxide anodized to anodic gallium oxide (AGO), tungsten oxide anodized to anodic tungsten oxide (AWO), zirconium oxide anodized to anodic zirconium oxide (AZO), and tin oxide anodized to anodic tin oxide (ASnO).

The layout or pattern may be characterized with respect to the density (number), porosity (pore area as a % portion of film area), pitch (center-point distance between neighboring wells of depressions 32 in nm), diameter (cell and/or well of depressions 32 in nm), and well depth (nm) of the depressions 32 in a defined area.

For example, the depressions 32 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 32 separated by less than about 100 nm, a medium density array may be characterized as having the depressions 32 separated by about 400 nm to about 1 μm, and a low-density array may be characterized as having the depressions 32 separated by greater than about 1 μm.

The layout or pattern of the depressions 32 may also or alternatively be characterized in terms of the average pitch, or the spacing from the center of one depression 32 to the center of an adjacent depression 32 (center-to-center spacing) or from the right edge of one depression 32 to the left edge of an adjacent depression 32 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In some embodiments, the depressions 32 are nanowells and have an average pitch (center-to-center spacing) about 250 nm or greater, 300 nm or greater, 350 nm or greater, 400 nm or greater, 450 nm or greater, 500 nm or greater, 550 nm or greater, 600 nm or greater, 650 nm or greater, or 700 nm or greater, or may be in a range between about 250 nm and 800 nm, 300 nm and 750 nm, 350 nm and 700 nm, 400 nm and 650 nm, 450 nm and 600 nm, 500 nm and 550 nm. In an example, the depressions 32 are nanowells and have an average pitch (center-to-center spacing) between about 350 nm and 750 nm. While example average pitch values have been provided, it is to be understood that other average pitch values may be used.

The depressions 32 may be characterized by the geometric shape of a cross-section of the depression 32 taken parallel to a predetermined plane, such as a face of the base support 22, or by the volume, opening area, depth, diameter, length, or width of the depression 32, or by a combination thereof. For example, the depressions 32 may be nanowells with a natural hexagonal morphology, or the depressions 32 may be nanowells with hexagonal openings, or the depressions 32 may be nanowells reconfigured as cylindrical structures with substantially circular openings. For another example, the opening area can range from about 1×10 3 μm² to about 100 μm², e.g., about 1×10 2 μm², about 0.1 μm², about 1 μm², at least about 10 μm², or more, or less. In another example, the volume can range from about 1×10⁻³ μm³ to about 100 μm³, e.g., about 1×10⁻² μm³, about 0.1 μm³, about 1 μm³, about 10 μm³, or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less. In another example, the depressions 32 are nanowells and the average depth is 150 nm or greater, 200 nm or greater, 250 nm or greater, 300 nm or greater, 350 nm or greater, or 400 nm or greater, or may be in a range between about 150 nm and 500 nm, 200 nm and 450 nm, or 250 nm and 400 nm, or 300 nm and 350 nm.

In one example, in addition to a single layer base support 14 and AMO film layer 16, the multi-layered structure 18 may include a coating (e.g., layer 34 in FIG. 1C) at the base of depressions 32 of a suitable low-background material, e.g., tantalum oxide (e.g., tantalum pentoxide or another tantalum oxide(s) (TaOx)) or another ceramic oxide at the surface, useful, e.g., as a solid support for molecular analysis. As another example, a polymeric resin may be applied to the base support 14. Suitable deposition techniques may include, e.g., chemical vapor deposition, dip coating, dunk coating, spin coating, spray coating, puddle dispensing, ultrasonic spray coating, doctor blade coating, aerosol printing, screen printing, and microcontact printing. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

The single layer base support 14 (whether used singly or as part of the multi-layered structure 18) may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a single base support 14 with any suitable dimensions may be used.

In an example, the flow channel 12 has a rectangular configuration. The length and width of the flow channel 12 may be selected so a portion of the single base support 14 or an outermost layer of the multi-layered structure 18 surrounds the flow channel 12 and is available for attachment to a lid (not shown) or another patterned or non-patterned structure. The surrounding portions are the bonding regions 26.

The depth of the flow channel 12 can be as small as a monolayer thick when microcontact, aerosol, or inkjet printing is used to deposit a separate material over the bonding region 26 that defines the flow channel 12 walls. In other examples, a thicker spacer layer may be applied to bonding region 26 so that the spacer layer defines at least a portion of the walls of the flow channel 12. As one example, the spacer layer can be a radiation-absorbing material that aids in bonding. In these examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

FIGS. 1B, 1C, and 1D depict examples of the architecture within the flow channel 12. The architecture shown in FIG. 1B is a patterned structure that includes depressions 22 and interstitial regions 24 defined in the AMO film layer 16 of the multi-layer structure 18, or alternatively or in addition to, the single base support 14. The architecture of the examples shown in FIGS. 1C and 1D is a patterned structure that includes functionalized pads 28 defined on surfaces 34 the single base support 14, or alternatively, on the layer 16 of the multi-layer structure 18. In one example, the interstitial regions 24 are non-functionalized, and substantially planer and featureless. In one example, the functionalized pads 28 are coextensive with respective surfaces 34. In another example, the functionalized pads 28 overlays a portion of respective surfaces 34.

For the patterned structure, many different layouts of the depressions 22 or functionalized pads 28 may be envisaged, including regular, repeating, and non-regular patterns. In an example, the depressions 22 or functionalized pads 28 are disposed in self-ordered hexagonal grid. As discussed in greater detail herein, other layouts may be engineered by pre-patterning processing using, for example, imprinting techniques, masking, photolithography, nanoimprint lithography (NIL), stamping techniques, embossing techniques, molding techniques, microetching techniques, or a combination thereof. for example, rectilinear (rectangular) layouts, triangular layouts, and so forth. In some examples, the layout or pattern can be an x-y lattice format in rows and columns. In other examples, the layout or pattern can be a repeating arrangement of depressions 22 or functionalized pads 28 and the interstitial regions 24. In still other examples, the layout or pattern can be a random arrangement of the depressions 22 within the interstitial regions 24.

The layout or pattern may be characterized with respect to the density (number) of the depressions 22 or functionalized pads 28 in a defined area. For example, the depressions 22 or functionalized pads 28 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 22 or functionalized pads 28 separated by less than about 100 nm, a medium density array may be characterized as having the depressions 22 or functionalized pads 28 separated by about 400 nm to about 1 μm, and a low density array may be characterized as having the depressions 22 or functionalized pads 28 separated by greater than about 1 μm.

The layout or pattern of the depressions 22 or functionalized pads 28 may also or alternatively be characterized in terms of the average pitch, or the spacing from the center of one depression 22 or functionalized pad 28 to the center of an adjacent depression 22 or functionalized pad 28 (center-to-center spacing) or from the right edge of one depression 22 or functionalized pad 28 to the left edge of an adjacent depression 22 or functionalized pad 28 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of can be between one of the lower values and one of the upper values selected from the ranges above. In an example, the depressions 22 have a pitch (center-to-center spacing) of about 1.5 μm. While example average pitch values have been provided, it is to be understood that other average pitch values may be used.

The size of each depression 22 may be characterized by its volume, opening area, depth, and/or diameter or length and width. For example, the volume can range from about 1×10-3 μm³ to about 100 μm³, e.g., about 1×10-2 μm³, about 0.1 μm³, about 1 μm³, about 10 μm³, or more, or less. For another example, the opening area can range from about 1×10-3 μm² to about 100 μm², e.g., about 1×10-2 μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

The size of each functionalized pad 28 may be characterized by its top surface area, height, and/or diameter or length and width. In an example, the top surface area can range from about 1×10-3 μm² to about 100 μm², e.g., about 1×10-2 μm², about 0.1 μm², about 1 μm², at least about 10 μm², or more, or less. For still another example, the height can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

Each of the architectures also includes a solid support 32 for staging molecular analyses herein. The solid support 32 includes the polymeric hydrogel and primers 36A, 36B. In the patterned structure of FIG. 1B, the solid support 32 is located within the depression 22. In the patterned structure of FIG. 1C, the solid support 32 is the functionalized pad 28. In the non-patterned structure of FIG. 1D, the solid support 32 extends along the lane 30.

Each of the architectures also includes the active area 32. The active area 32 may include a polymeric hydrogel and primers 36A, 36B. In the patterned structure of FIG. 1B, the active area 32 is located within the depression 22. In the patterned structure of FIG. 1C, the active area 32 is the functionalized pad 28.

The polymeric hydrogel may be any gel material that can swell when liquid is taken up and can contract when liquid is removed, e.g., by drying. In an example, the polymeric hydrogel includes an acrylamide copolymer, such as poly(N-(5-azidoacetamidylpentyl) acrylamide-co-acrylamide, PAZAM or other forms of the acrylamide copolymer In some examples, PAZAM and other forms of the acrylamide copolymer are linear polymers. In some other examples, PAZAM and other forms of the acrylamide copolymer are lightly cross-linked polymers. In other examples, the gel material may be a variation of the structure (I). In one example, the acrylamide unit may be replaced with N,N-dimethylacrylamide.

It is to be understood that other polymeric hydrogels 34 may be used, as long as they are functionalized to graft oligonucleotide primers 36A, 36B thereto. Some examples of suitable the polymeric hydrogel include functionalized polysilanes, such as norbornene silane, azido silane, alkyne functionalized silane, amine functionalized silane, maleimide silane, or any other polysilane having functional groups that can attach the desired primer set 36A, 36B. Other examples of suitable polymeric hydrogels 34 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels include mixed copolymers of acrylamides and acrylates. A variety of polymer architectures containing acrylic monomers (e.g., acrylamides, acrylates etc.) may be utilized in the examples disclosed herein, such as branched polymers, including dendrimers, and the like. For example, the monomers (e.g., acrylamide, etc.) may be incorporated, either randomly or in block, into the branches (arms) of a dendrimer.

The polymeric hydrogel may be formed using any suitable copolymerization process. The polymeric hydrogel may be deposited using any of the methods disclosed herein. For at least some of the deposition techniques, the polymeric hydrogel may be incorporated into a mixture, e.g., with water or with ethanol and water, and then applied. The attachment of the polymeric hydrogel to the underlying base support 14 or AMO layer 16 of the multi-layer structure 18 may be through covalent bonding. In some instances, the underlying base support 14 or layer 16 may first be activated, e.g., through silanization or plasma ashing. As discussed in greater detail herein, with respect to certain embodiments herein, each of the architectures also includes the primer 36A, 36B attached to the polymeric hydrogel.

### Example AMO Supported Microarray on an Image-Generating Chip

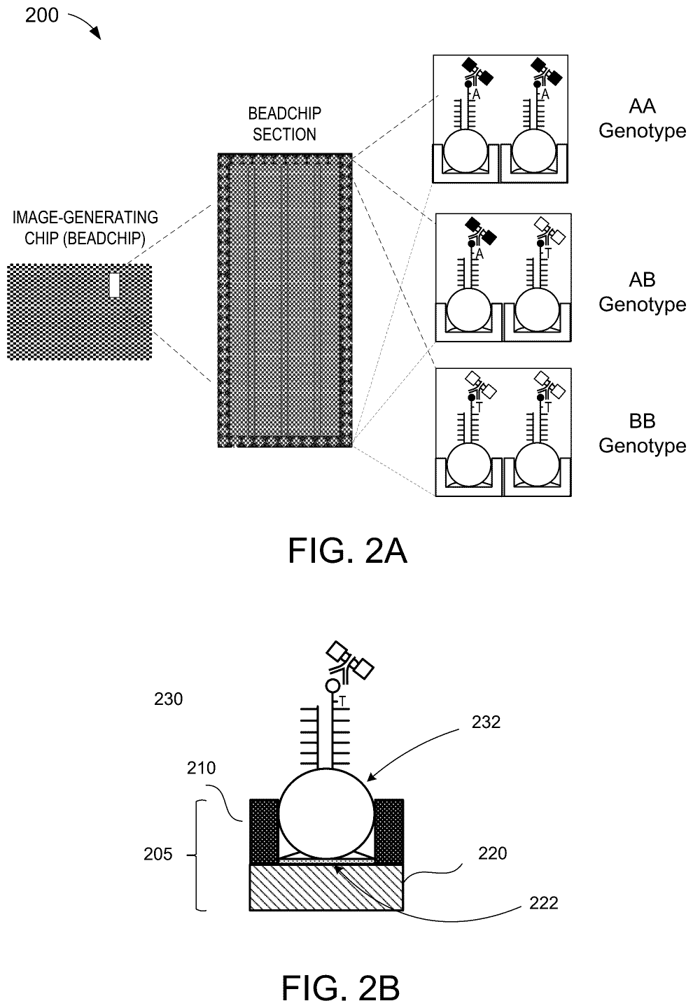

An example of an AMO-Supported bead-based microarray on an image-generating chip platform (i.e., BeadChip or BeadArray) is shown in FIG. 2, an illustration 200 indicating capture probes 201 that may be positioned on an image-generating chip 208 in a genotyping device. The image-generating chip 208 may also referred to as BeadChips or BeadArrays. The image-generating chip 208 may include multiple sections 207 that are arranged in columns and rows on the image-generating chip 208. For example, the image-generating chip 208 may have 12, 24, 96 or more sections, each of which may have a separate DNA sample. Beads 204 (e.g., glass beads) may be positioned in wells 206 (or holes) at known locations on the surface of the image generating chip 208. Numerous (e.g., thousands or more) different types of oligonucleotide probes 201 may be attached to the beads 204 at random known locations and replicated many times (up to 10 times or more) on the image-generating chip 208. In some implementations the Illumina Infinium™ platform may be used, which includes hundreds of thousands to millions of micro-wells on a BeadChip, and microbeads are distributed in the microwells. The microbeads have diameters of roughly 3 μm. DNA samples are processed, amplified, and provided to the BeadChip. Each bead 204 is covered with hundreds of thousands of copies of a specific oligonucleotide that acts as capture sequences targeting different SNPs. Allelic specificity of hybridized DNA 202 is conferred by enzymatic base extension at 3′ end of the probe. Base extensions are applied fluorescent labels 203, imaged under excitation, and allele signal intensity data is used to perform genotype calling.

Similar to flow cells as illustrated, e.g., in FIGS. 1B through 1D, the image generating chip 208 may include a multi-layered or composite structure 205, which includes, a minimum, a single layer base support 222 overlayed with an AMO film layer 210. The surface of the AMO film layer 210 imparted with an array of microwells 232, bottom portions of which are covered in functional pads 230. Similar to example flow cells herein, the thickness of the AMO film layer may be coterminous with the depth of the microwells 232, such that a base portion of the inner surface of the microwells exposes a surface 222 of the single layer base supports 220. Alternatively, the thickness of the AMO film layer may accommodate the entire depth of the microwell, such the entire inner surface of the microwell is formed in the material of the AMO film layer. The functional pads 230 may include one or more primers for immobilizing probe-functionalized microbeads.

### Preparation of AMO-Based Substrates Supported on Flow Cells and Other Devices

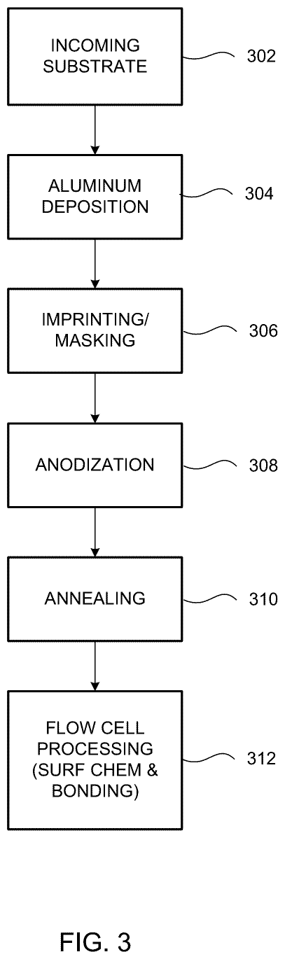

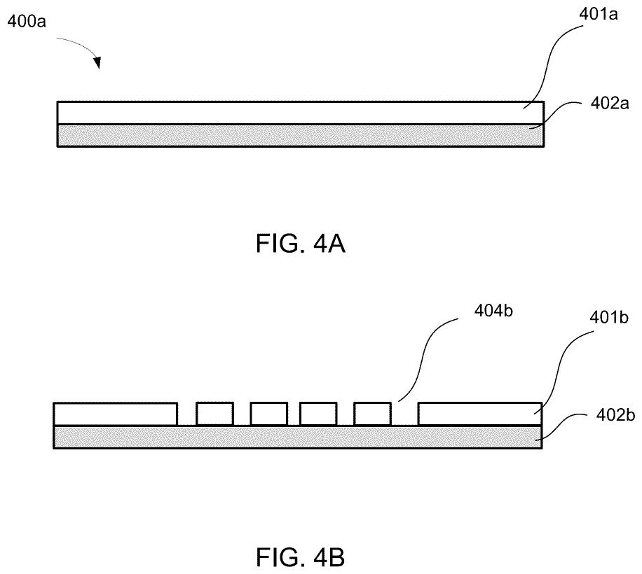

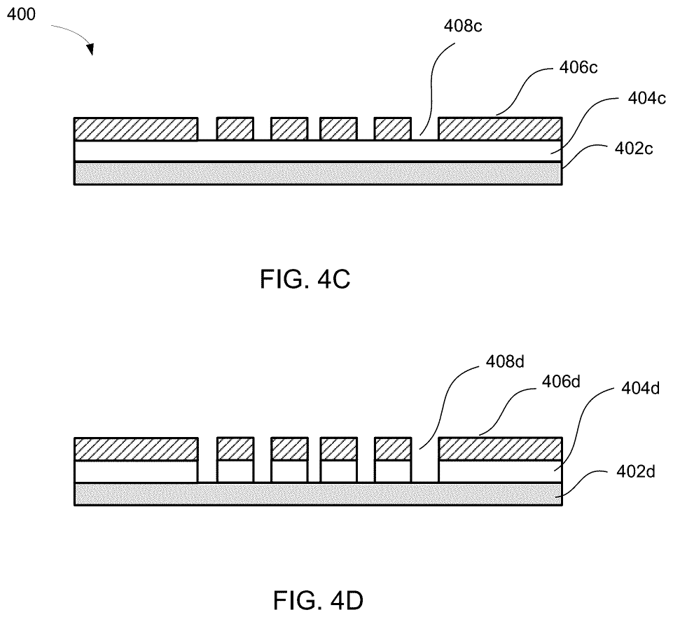

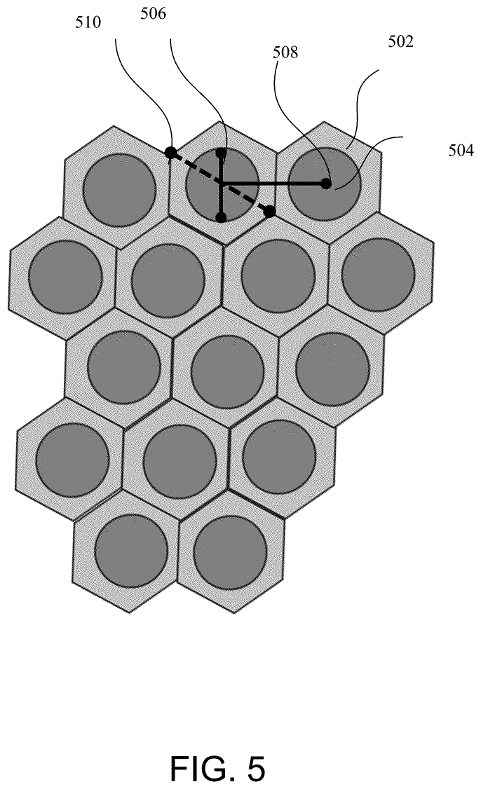

Examples of the methods described herein may be used in the preparation of AMO-based substrates supported on flow cells and other apt devices. An exemplary process for making a flow channel supporting an AAO film layer having a patterned structure of well-ordered nanowells is shown in FIGS. 3 and 4A-4D. The example process is adapted for automated manufacture of flow cells herein at production scale. However, the process can also be adapted, e.g., for laboratory production of flow cells, which is also contemplated herein.

Referring back to FIGS. 1B and 1C, the flow cell prepared according to the example method includes multi-layered or composite structure 18, which includes, a minimum, a single layer base support layer 14 overlayed with an AAO film layer 16. The surface of the AMO film layer 16 is imparted with an array of depressions 32, bottom portions of which are covered in functional pads 20. The AAO film layer 16 is coterminous with the depth of the depressions 32—nanowells in the example—such that a base portion of the inner surface of the depressions exposes a surface 34 of the single layer base support 14, which serves as a functionalized, low-background solid phase support for molecular analysis.

Referring to FIG. 3, an incoming substrate 302 forms the single layer base support 404 (shown in FIG. 4A), which may a be glass or silicon wafer, or glass panel, with a thickness between ˜200 nm to 300 nm, although substrates of less than 200 nm or greater than 300 nm, depending on the particular requirements of a given application, are also contemplated. During an aluminum deposition 304, an aluminum film 702 may be deposited on the single layer base support 404 using a suitable technique, such as chemical vapor deposition, dip coating, dunk coating, spin coating, spray coating, puddle dispensing, ultrasonic spray coating, doctor blade coating, aerosol printing, screen printing, microcontact printing, or by other techniques known in the art. The thickness of the deposited aluminum film is selected to correspond with the desired depth of the depressions 32, as discussed. For NGS and other sequencing applications, films having a thickness anywhere from 200 nm to 500 nm are adequate for most applications. After deposition, the aluminum film 402 may be cleaned ultrasonically and/or with organic solvent to remove grease residues from its surface. Then the cleaned aluminum film 402 may be washed with an alkaline chemical liquid, such as sodium carbonate, to achieve a degree of surface etching. The surface etched aluminum foil may also be subjected to a neutralization step, such as treatment with nitric acid, to remove excess alkali.

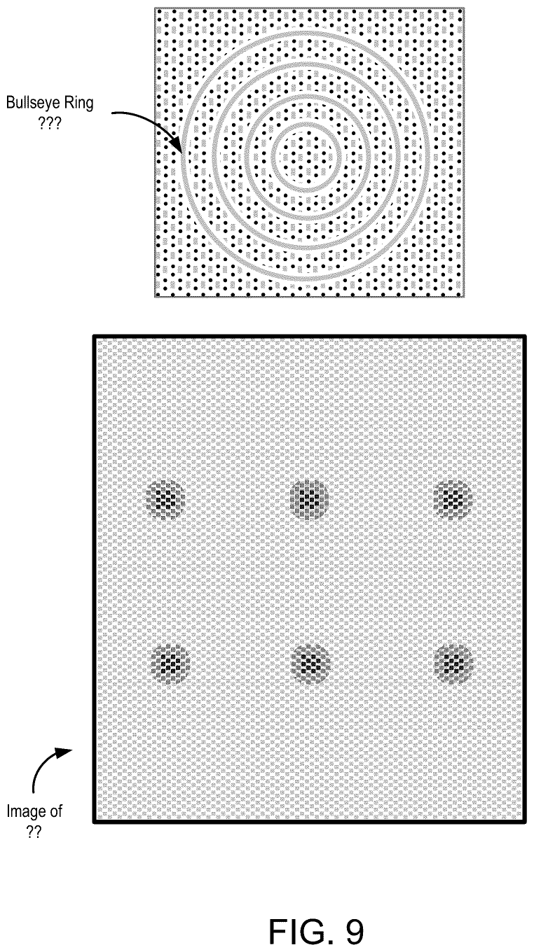

During imprinting/marking 305, a traditional NIL imprint layer may be added to the aluminum film surface to define one or more long range macroscopic features (e.g., unpatterned interstitial areas separating patterned lanes, registration fiducials, alignment features). An example of a fiducial is illustrated in FIG. 9. A fiducial may be is a distinguishable point of reference in or on an object, e.g., the point of reference is present in an image of the object, is present in a data set derived from detecting the object, or any other representation of the object suitable to express information about the point of reference with respect to the object. The point of reference is specifiable by an x and/or y coordinate in a plane of the object. Alternatively, or additionally, the point of reference is specifiable by a z coordinate that is orthogonal to the x-y plane, e.g., being defined by relative locations of the object and a detector. One or more coordinates for a point of reference are specifiable relative to one or more other features of an object or of an image or other data set derived from the object.

Optionally, during imprinting/marking 305, the aluminum film is mechanically stamped, or laser etched, otherwise imparted with a pre-determined pattern of dimples or pits in the metal oxide file, which serve as nucleation sites to the guide the growth of nanowells. (See Lang et al. Nanoscale Advances 3 (10), pp. 2918-2923 and Kustandi et al. ACS nano 4 (5), pp. 2561-2568, each of which is incorporated here in its entirety.)

Importantly, AMO surface morphology may be calibrated to the particular operation, support format, or imaging equipment for a given molecular analysis, based on manipulation of process parameters during anodization 306. Pre-patterning allows pre-tuning of nanowell geometries either within the natural honeycomb pattern forming during anodization 306, or any number of non-hexagonal latticework patterns, suitable for use herein, including, e.g., square, triangular, diamond and hybrid patterns.

A number of pre-patterning techniques are contemplated herein, including hard stamping using, e.g., an SiC stamp, Ni Stamp, or SI₂N₄ stamp; focused ion beam (FIB) lithography, holographic lithography, resist-assisted FIB lithography, colloidal lithography, block copolymer self-assembly, soft imprinting, nanoimprint lithography, and step and flash lithography. Certain techniques are particularly amenable to production scale batch microfabrication, including, e.g., nanoimprint lithography, as described in Lang et al. Nanoscale Adv., 2021, 3, 2918-2923, and step and flash lithography, as disclosed in Kustandi et al, ACSNano Vol. 4, No. 5, 2561-68, each of which is incorporated herein in its entirety.

Step and Flash Imprint Lithography employs a quartz template fabricated using conventional electron beam lithography and etching process, which renders pillar points of the template in any number of patterns, diameters, depths, pitches, any of which may be selected based on a desired AAO morphology. (See W. Lee et al. Chem. Rev. 2014, 114, 15, 7487-7556, which is incorporated by reference herein in its entirety.) A subject aluminum-coated glass substrate optionally may be pretreated with an etch barrier, e.g., with a planarization coating of Transpin HE-0600 (Molecular Imprints, Inc.). The quartz template is brought into contact with substrate, which displaces a UV-polymerizable imprint resist material (e.g., liquid acrylate) dispersed across the surface of the aluminum. After the imprint resist is polymerized under UV light, the template is removed leaving an exact inverse replica of the template in the imprint resist and exposed aluminum surface from which the imprint resist is displaced. After imprinting, the sample may then be exposed to oxygen plasma, e.g., in a RIE Oxford Etcher, Plasmalab ICP180 under appropriate conditions, etching a pattern of pits in the exposed aluminum surface, which then serve as nucleation sites to guide the growth of nanowells during anodization.

In another example pre-patterning technique is application of a sacrificial template layer generated using nanoimprint lithography (NIL), which is a relatively simple nanolithography process with low cost, high throughput, and high resolution. Similar to the Step and Flash Imprint Lithography above, NIL creates patterns by mechanical deformation of imprint resist and subsequent processes.

AMO may be fabricated by anodization 306 under either constant potential (i.e., potentiostatic) or a constant current (i.e., galvanostatic) condition. Referring to FIG. 5, AMO growth during anodization 306 may be measured according to any one more of cell diameter 510 of cells 502/pore diameter 560 of pores 504, pitch 508, and porosity, a percentage portion of the total area of pores 504 to the total area of the patterned surface (here, the total area of cells 502). AMO growth of ordered nanowells generally occurs within a narrow range of tunable, interdependent anodizing conditions, which differ depending on the acid selection. For example, the optimum anodizing potential (U) for ordering of nanowells using 0.3 M sulfuric acid is at U=25 V, which results in a pitch of about 65 nm; using 0.3 M oxalic acid is at U=40 V, which results in a pitch of about 103 nm; using 0.3 M selenic acid is at U=48 V for a pitch of about 112 nm; and using 0.3 M phosphoric acid is at U=195 V for a pitch of about 500 nm. In addition to the type of acid, tunable conditions that affect anodic oxidation and AMO film morphology include (a) anodization applied voltage (Vap) or current, (b) the concentration of the acidic electrolyte, and (c) temperature (T).

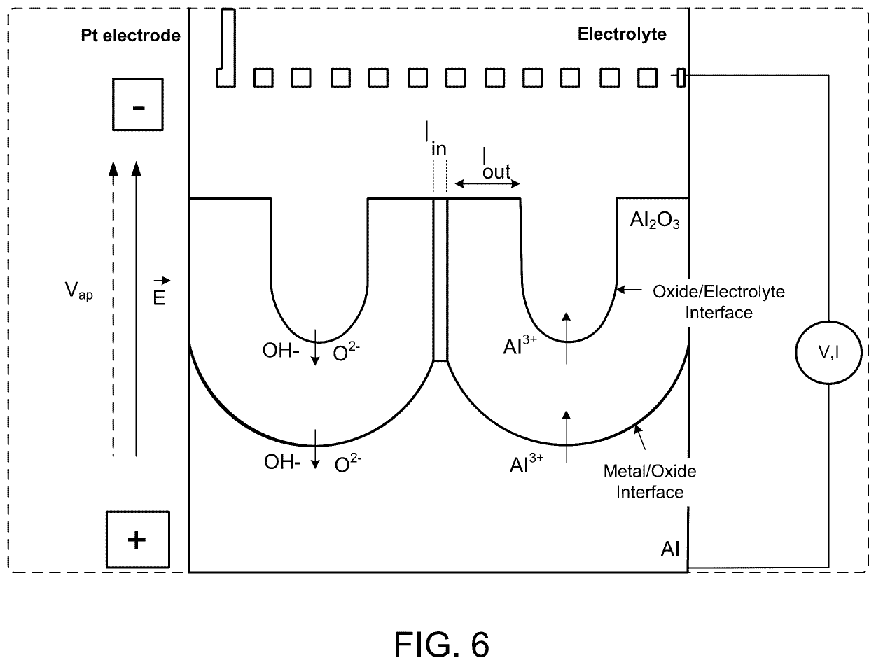

Referring to FIG. 6, AMO growth depends on a balance between electrical-field-driven oxide formation at the metal/oxide interface 602 and oxide dissolution at the electrolyte/oxide interface 604. The occurring electrochemical processes are expressed as

2Al(s)+3H₂O(l)→Al₂O₃(s)+6H+(aq)+6e⁻ (1)

Al2O3(s)+6H⁺(aq)→2Al₃⁺(aq)+3H₂O(l) (2)

2H+(aq)+2e⁻→H₂(g) (3)

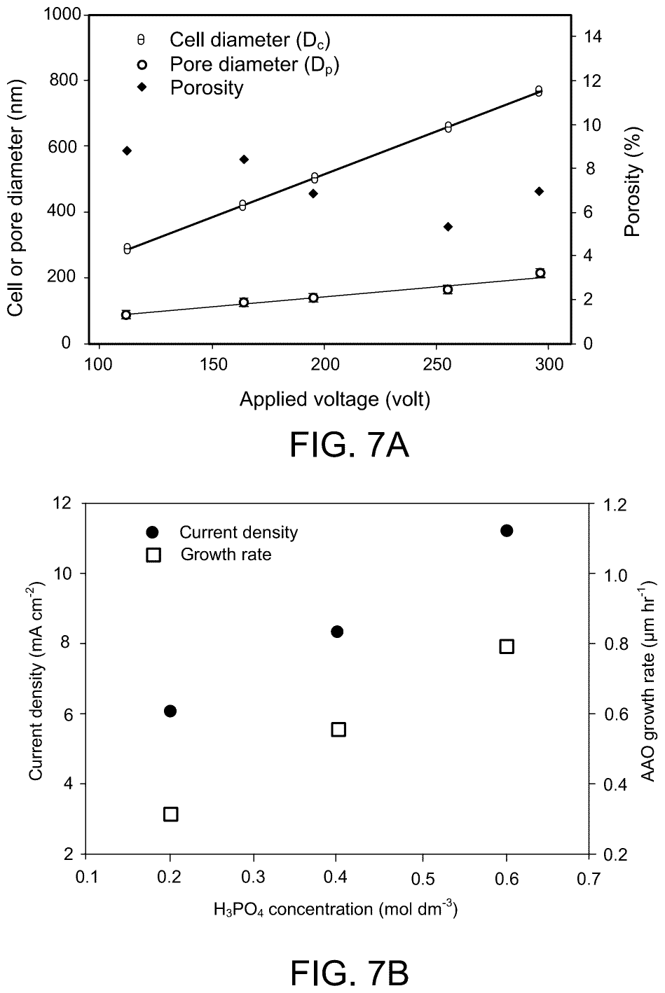

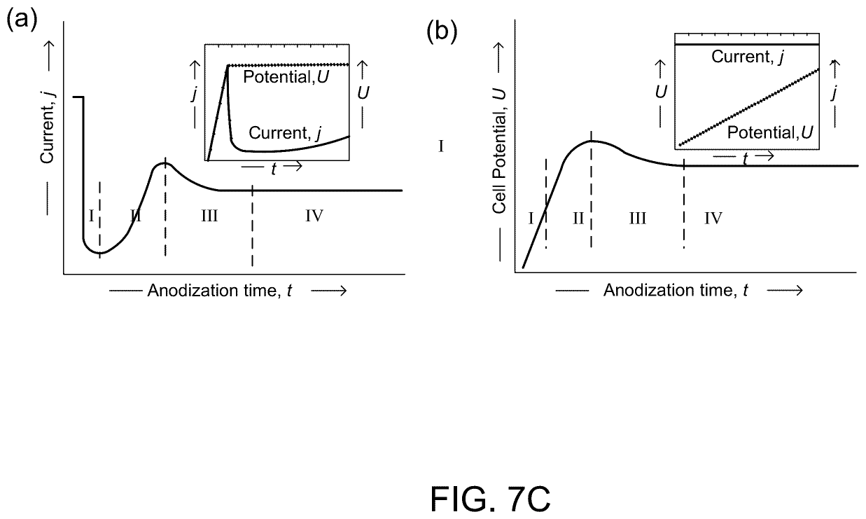

which corresponds to the formation (1) and dissolution (2) of the oxide at the anode 606, while hydrogen is released (3) at the cathode 608. The AMO growth in terms of cell size and pore size involves movements of ionic species (i.e., ion mobility or flux) between electrolyte/oxide and oxide/metal interfaces under an electric field. AMO growth increases linearly with anodization voltage, where the relationship between voltage and cell size is governed through the relationship Dc≈Va×2.6 nm/V, and the relationship of pore size and voltage is governed by the relationship is Dp≈Va×0.6 nm/volt. The anodization current is related to the electrolyte concentration, electric field, and temperature according to the following equation:

$$i = 2 anv exp ( - ( \frac{W - qaE}{kT} ) )$$

where n is the ion concentration, v is the jump frequency, a is the activation distance, W is the energy barrier and E is the interfacial electric field. Exponential dependence of anodization current on the electrical field dictates the exponential relationship between AMO growth rate and acidic electrolyte concentration. The influence of applied voltage to cell/pore diameter and porosity and acidic electrolyte concentration to current density and AMO (AAO) growth rate are plotted in FIGS. 7A and 7B, respectively.

The AMO growth rate bears a linear relationship with ion mobility or flux, as measured by current density. As illustrated in the graph of FIG. 7C, whether performed under potentiostatic or galvanostatic conditions, the current density, and thus the AMO growth rate, are highest near start of the anodization process, and decrease over time until a steady state is reached (stage III). Generally, AMO growth of ordered nanowells generally occurs only within the narrow window of stage III, after which AMO growth becomes morphologically unstable. Here due to the constant anodic potential applied during anodization 306, a thin compact barrier oxide starts to grow over the entire aluminum surface (stage I). Thickening of the initial barrier oxide over time (t) results in an increase of the series resistance (R) of the anodization circuit. Current (j) is initially maintained at the limiting current (jₗᵢₘᵢₜ) of the power supply, and correspondingly potential (U=jR) increases linearly with time (t). When the thickness (or the resistance, R) of the compact barrier oxide layer reaches a certain value, current (j) drops rapidly to hit a steady-state value (stage IV), at which time the current concentrates on aberrant loci existing on the initial barrier oxide, resulting in non-uniform oxide thickening and pore initiation at the thinner oxide areas.

Moreover, current density and AMO growth rate increases exponentially with the acid concentration over processing time until a critical value is reached, at which the anodization current density surges and AMO growth becomes nonuniform or breaks down. Increasing anodization voltage decreases the critical value of acid concentration leading to breakdown. Thus, as the cell size scales with the voltage, fabricating a nanoporous AAO film presents challenges due to breakdown.

Both to avoid critical breakdown and increase the period of AMO growth of ordered nanowells during relative mild conditions, in some embodiments, the anodization process 306 may be staged in a two-step process. In one example, the first anodization process is performed through steady stead for an extended period of time (10 or more hours) and, preferably, under mild anodization conditions. The resulting nanostructure of the AAO film includes pore structures having an extended depth with disordered portions adjacent the pore openings representing AAO growth during extended steady state period (Stage IV). The disordered upper portions are then removed by a PC etching process using, for example, an aqueous mixture of 0.5

M H₃PO₄ and 0.2 M CrO₃ at 80° C. The surface of the resulting AAO is textured with arrays of hemispherical features similar to pits formed in a pre-patterning technique, discussed herein. A second anodization is then carried out with the textured AAO under the same or similar conditions employed for the first anodizing step, in which well-ordered pores nucleate at the centers of each concave feature.

One or more additional anodizing steps may be performed to further widen nanowell pores to desired size, as described, e.g., in Rahman et al. Nanoscale research letters 7(1), pp. 1-7, which is incorporated by reference herein in its entirety.

After completion of anodization 306, the AAO film may optionally be subject to annealing 610, which imparts stability in the film under acidic and basic conditions common to the flow cell applications contemplated herein and, in addition, can reduce defect driven photoluminescence to ensure low background noise of the material. (See Mardilovich et al. Journal of membrane science 98 (1-2), pp. 143-155, Stojadinovic et al. Applied Surface Science 256 (3), pp. 763-767, Sun et al. Journal of luminescence 121 (2), pp. 588-594, and Roslyakov et al. Surface and Coatings Technology, 381, p. 125159, each of which is incorporated herein in its entirety.)

In one example, the anodized aluminum oxide is annealed in air for about four (4) hours at a temperature of from about 200° C. to up to 1200° C., and preferably from about 600° C. to up to 1000° C. Alternatively, the anodized aluminum oxide may be annealed in an inert atmosphere, such as under argon, for about six (6) hours at a temperature of from about 200° C. to up to 800° C. For NGS applications, transparency of the anodic oxides should also be considered, particularly for the 300-1000 nm wavelength range. For AAO films having a thickness of from nine microns to 45 microns, annealing at 800° C. resulted in the highest percentage of transmission in the desired wavelength range. For example, nine-micron thick films annealed at 800° C. were about 90% transmissive across a range of 300-1000 nm. It is anticipated that thinner films will demonstrate transparency similar to or better than the nine-micron specimens. Importantly, with annealing, and the anodic oxidation processing of the alumina itself, the stress in the film must be considered to prevent excessive bending moments, or in the extreme, delamination or other stress relieving behavior, as such stress may change characteristics of the pore structure of the nanowells. (See Liao et al. Corrosion science 74, pp. 232-239, which is incorporated by reference herein in its entirety.)

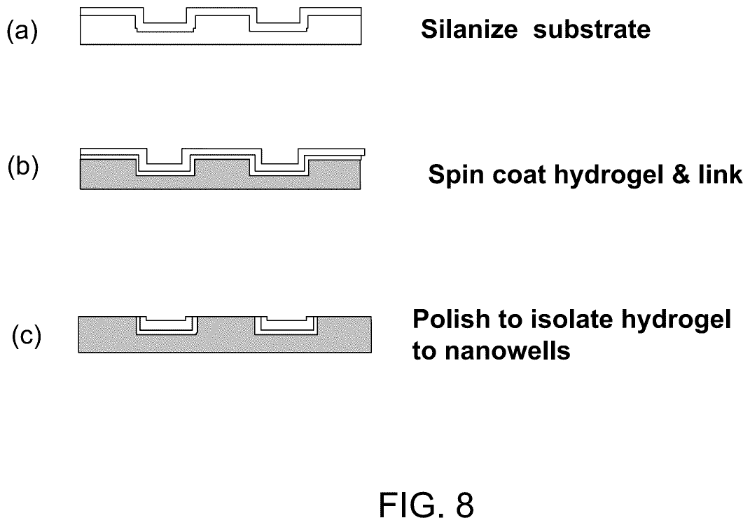

After completion of anodization 306, including, optionally, annealing 610, the substrate is put through a traditional flow cell process workflow. For example, the solid support 32 may provide a surface chemistry including a functionalized coating layer and one or more surface primers covalently bound to the functionalized coating layer to create a reaction site for a biological sample. FIG. 8 depicts the selective application of a polymer coating layer 802 into each of the depressions 804a and 804b. The selective application of the polymer 802 may involve multiple processes, including activation of the interstitial regions 806 and the exposed surfaces in the depressions 804a and 804b, depositing the polymer 802 on the activated interstitial regions 806 and in the depressions 804a and 804b, and polishing the interstitial regions 806 to remove polymer 802 from the interstitial regions to limit polymer 802 to depressions 804a and 804b.

In some examples, activation involves silanizing the surface, including the interstitial regions 806 and the surfaces of depressions 804a and 804b of the support 800. Silanization may be accomplished using any silane or silane derivative. The selection of the silane or silane derivative may depend, in part, upon the polymer 802 that is to be formed, as it may be desirable to form a covalent bond between the silane or silane derivative and the polymer 802. The method used to attach the silane or silane derivative may vary depending upon the silane or silane derivative that is being used. Several examples are set forth herein.

Examples of suitable silanization methods include vapor deposition (e.g., a YES method), spin coating, or other deposition methods. In an example utilizing the YES CVD oven, the support 800 is placed in the CVD oven. The chamber may be vented and then the silanization cycle started. During cycling, the silane or silane derivative vessel may be maintained at a suitable temperature (e.g., about 120° C. for norbornene silane), the silane or silane derivative vapor lines be maintained at a suitable temperature (e.g., about 125° C. for norbornene silane), and the vacuum lines be maintained at a suitable temperature (e.g., about 145° C.).

In another example, the silane or silane derivative (e.g., liquid norbornene silane) may be deposited inside a glass vial and placed inside a glass vacuum desiccator with the support 800. The desiccator can then be evacuated to a pressure ranging from about 15 mTorr to about 30 mTorr, and placed inside an oven at a temperature ranging from about 60° C. to about 125° C. Silanization is allowed to proceed, and then the desiccator is removed from the oven, cooled and vented in air.

Vapor deposition, the YES method and/or the vacuum desiccator may be used with a variety of silane or silane derivatives, such as those silane or silane derivative including a cycloalkene unsaturated moiety, such as norbornene, a norbornene derivative (e.g., a (hetero) norbornene including an oxygen or nitrogen in place of one of the carbon atoms), transcyclooctene, transcyclooctene derivatives, transcyclopentene, transcycloheptene, trans-cyclononene, bicyclo[3.3.1]non-1-ene, bicyclo[4.3.1]dec-1 (9)-ene, bicyclo[4.2.1]non-1 (8)-ene, and bicyclo[4.2.1]non-1-ene. Any of these cycloalkenes can be substituted, for example, with an R group, such as hydrogen, alkyl, alkenyl, alkynyl, cycloalkyl, cycloalkenyl, cycloalkynyl, aryl, heteroaryl, heteroalicyclic, aralkyl, or (heteroalicyclic) alkyl. An example of the norbornene derivative includes [(5-bicyclo [2.2.1]hept-2-enyl) ethyl]trimethoxysilane. As other examples, these methods may be used when the silane or silane derivative includes a cycloalkyne unsaturated moiety, such as cyclooctyne, a cyclooctyne derivative, or bicyclononynes (e.g., bicyclo[6.1.0]non-4-yne or derivatives thereof, bicyclo[6.1.0]non-2-yne, or bicyclo[6.1.0]non-3-yne). These cycloalkynes can be substituted with any of the R groups described herein.

The attachment of the silane or silane derivative forms an activated surface, both on the interstitial regions 806 and in the depressions 804a and 804b.

The polymer layer 802 may then be applied as described herein. As examples, the polymer (e.g., PAZAM) may be deposited using spin coating, or dipping or dip coating, or flow of the functionalized molecule under positive or negative pressure, or another suitable technique. The polymer deposited to form the polymer layer 802 may be present in a mixture. In an example, the mixture includes PAZAM in water or in an ethanol and water mixture.

After being coated, the mixture including the polymer may also be exposed to a curing process to form the polymer layer 802 across the activated interstitial regions 30 of the patterned resin 54′ and in the depressions 408a and 408b. In an example, curing may take place at a temperature ranging from room temperature (e.g., about 25° C.) to about 95° C. for a time ranging from about 1 millisecond to about several days. In another example, the time may range from 10 seconds to at least 24 hours. In still another example, the time may range from about 5 minutes to about 2 hours.

The attachment of the polymer layer 802 to the activated (in this example silanized) surfaces may be through covalent bonding. Covalent linking is helpful for maintaining at least the first primer set in the depressions 804a and 804b throughout the lifetime of the ultimately formed flow cell during a variety of uses. The following are some examples of reactions that can take place between the activated (e.g., silanized) surfaces and the polymer layer 802.

When the silane or silane derivative includes norbornene or a norbornene derivative as the unsaturated moiety, the norbornene or a norbornene derivative can: i) undergo a 1,3-dipolar cycloaddition reaction with an azide/azido group of PAZAM; ii) undergo a coupling reaction with a tetrazine group attached to PAZAM; undergo a cycloaddition reaction with a hydrazone group attached to PAZAM; undergo a photo-click reaction with a tetrazole group attached to PAZAM; or undergo a cycloaddition with a nitrile oxide group attached to PAZAM.

When the silane or silane derivative includes cyclooctyne or a cyclooctyne derivative as the unsaturated moiety, the cyclooctyne or cyclooctyne derivative can: i) undergo a strain-promoted azide-alkyne 1,3-cycloaddition (SPAAC) reaction with an azide/azido of PAZAM, or ii) undergo a strain-promoted alkyne-nitrile oxide cycloaddition reaction with a nitrile oxide group attached to PAZAM.

When the silane or silane derivative includes a bicyclononyne as the unsaturated moiety, the bicyclononyne can undergo similar SPAAC alkyne cycloaddition with azides or nitrile oxides attached to PAZAM due to the strain in the bicyclic ring system.

In other examples, plasma ashing rather than silanization may be used to activate the interstitial regions 806 and the exposed surfaces of the support 800 in the depressions 808a and 808b. After plasma ashing, the mixture containing the polymer may be directly spin coated (or otherwise deposited) on the plasma ashed surfaces and then cured to form the polymer layer 32. In this example, plasma ashing may generate surface-activating agent(s) (e.g., hydroxyl (C—OH or Si—OH) and/or carboxyl groups) that can adhere the polymer to the interstitial regions 806 and the exposed surfaces of the support 800 in the depressions 804a and 804b. In these examples, the polymer 802 is selected so that it reacts with the surface groups generated by plasma ashing.

In some sequencing operations involving target nucleic acid materials, the primers may be an oligonucleotide (i.e., a surface oligo) having a complementary sequence to the target nucleic acid material, in which case the target nucleic acid material may be captured or immobilized at the reaction site through hybridization to a primer under stringent conditions. A primer may be functionalized with a capture agent, e.g., streptavidin, and the nucleic acid sequence is ligated with a capture partner, e.g., biotin, (or vice versa) in which case the target nucleic acid material may be captured at the reaction site through, e.g., formation of a streptavidin-biotin complex. The capture agent may also be ligated to the functionalized coating layer and capture takes place without the need for a primer. The target nucleic acid material may also be captured through a reaction of clickable groups conjugating either the nucleic acid material and a primer or nucleic acid material and a functionalized coating layer.

Flow Cells with Image Sensors

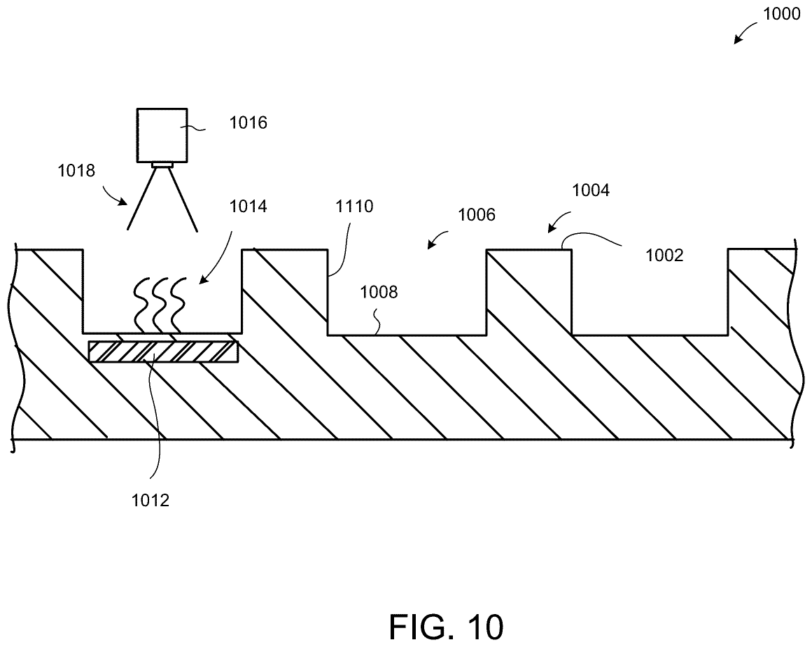

FIG. 10 shows a portion of a channel within a flow cell 1000 that is an example of a variation of the flow cell 1000. In other words, the channel depicted in FIG. 10 is a variation of the flow channel 1010 of the flow cell 1000. This flow cell 1000 is operable to read polynucleotide strands 1014 that are secured to the floor 1008 of wells 1006 in the flow cell 1000. By way of example only, the floor 1008 where polynucleotide strands 1014 are secured may include a co-block polymer capped with azido. By way of further example only, such a polymer may comprise a poly(N-(5-azidoacetamidylpentyl) acrylamide-co-acrylamide) (PAZAM) coating provided in accordance with at least some of the teachings of U.S. Pat. No. 9,012,022, entitled “Polymer Coatings,” issued Apr. 21, 2015, which is incorporated by reference herein in its entirety. Such a polymer may be incorporated into any of the various flow cells described herein.

In the present example, the wells 1006 are separated by interstitial spaces 1014 provided by the base surface 1012 of the flow cell 1000. Each well 1030 has a sidewall 1008 and a floor 1010. The flow cell 1000 in this example is operable to provide an image sensor 1040 under each well 1030. In some versions, each well 1030 has at least one corresponding image sensor 1040, with the image sensors 1040 being fixed in position relative to the wells 1030. Each image sensor 1040 may comprise a CMOS image sensor, a CCD image sensor, or any other suitable kind of image sensor. By way of example only, each well 1030 may have one associated image sensor 1040 or a plurality of associated image sensors 1040. As another variation, a single image sensor 1040 may be associated with two or more wells 1030. In some versions, one or more image sensors 1040 move relative to the wells 1030, such that a single image sensor 1040 or single group of image sensors 1040 may be moved relative to the wells 1030. As yet another variation, the flow cell 1000 may be movable in relation to the single image sensor 1040 or single group of image sensors 1040, which may be at least substantially fixed in position.

Each image sensor 1040 may be directly incorporated into the flow cell 1000. Alternatively, each image sensor 1040 may be directly incorporated into a cartridge such as the removable cartridge 200, with the flow cell 1000 being integrated into or otherwise coupled with the cartridge. As yet another illustrative variation, each image sensor 1040 may be directly incorporated into the base instrument 1002 (e.g., as part of the detection assembly 1010 noted above). Regardless of where the image sensor(s) 1040 is/are located, the image sensor(s) 1040 may be integrated into a printed circuit that includes other components (e.g., control circuitry, etc.). In versions where the one or more image sensors 1040 are not directly incorporated into the flow cell 1000, the flow cell 1000 may include optically transmissive features (e.g., windows, etc.) that allow the one or more image sensors 1040 to capture fluorescence emitted by the one or more fluorophores associated with the polynucleotide strands 550 that are secured to the floors 534 of the wells 1030 in the flow cell 1000 as described in greater detail below. It should also be understood that various kinds of optical elements (e.g., lenses, optical waveguides, etc.) may be interposed between the floors 534 of the wells 1030 and the corresponding image sensor(s) 1040.

As also shown in FIG. 10, a light source 1060 is operable to project light 562 into the well 1030. In some versions, each well 1030 has at least one corresponding light source 1060, with the light sources 1060 being fixed in position relative to the wells 1030. By way of example only, each well 1030 may have one associated light source 1060 or a plurality of associated light sources 1060. As another variation, a single light source 1060 may be associated with two or more wells 1030. In some other versions, one or more light sources 1060 move relative to the wells 1030, such that a single light source 1060 or single group of light sources 1060 may be moved relative to the wells 1030. As yet another variation, the flow cell 1000 may be movable in relation to the single light source 1060 or single group of light sources 1060, which may be substantially fixed in position. By way of example only, each light source 1060 may include one or more lasers. In another example, the light source 1060 may include one or more diodes.

Each light source 1060 may be directly incorporated into the flow cell 1000. Alternatively, each light source 1060 may be directly incorporated into a cartridge such as the removable cartridge, with the flow cell 1000 being integrated into or otherwise coupled with the cartridge. As yet another illustrative variation, each light source 1060 may be directly incorporated into the base instrument 1002. In versions where the one or more light sources 1060 are not directly incorporated into the flow cell 1000, the flow cell 1000 may include optically transmissive features (e.g., windows, etc.) that allow the wells 1030 to receive the light emitted by the one or more light source 1060, to thereby enable the light to reach the polynucleotide strands 1050 that are secured to the floor 534 of the wells 1030. It should also be understood that various kinds of optical elements (e.g., lenses, optical waveguides, etc.) may be interposed between the wells 1030 and the corresponding light source(s) 1060.

As described elsewhere herein, a DNA reading process may begin with performing a sequencing reaction in the targeted well(s) 1030 (e.g., in accordance with at least some of the teachings of U.S. Pat. No. 9,453,258, entitled “Methods and Compositions for Nucleic Acid Sequencing,” issued Sep. 27, 2016, which is incorporated by reference herein in its entirety). Next, the light source(s) 1060 is/are activated over the targeted well(s) 1030 to thereby illuminate the targeted well(s) 1030. The projected light 562 may cause a fluorophore associated with the polynucleotide strands 1050 to fluoresce. Accordingly, as shown in block 594 of FIG. 6, the corresponding image sensor(s) 1040 may detect the fluorescence emitted from the one or more fluorophores associated with the polynucleotide strands 1050. A system controller of the base instrument 1002 may drive the light source(s) 1060 to emit the light. The system controller of the base instrument 1002 may also process the image data obtained from the image sensor(s) 1040, representing the fluorescent emission profiles from the polynucleotide strands 1050 in the wells 1030. Using this image data from the image sensor(s) 1040 the system controller may determine the sequence of bases in each polynucleotide strand 1050. By way of example only, this process and equipment may be utilized to map a genome or otherwise determine biological information associated with a naturally occurring organism, where DNA strands or other polynucleotides are obtained from or otherwise based on a naturally occurring organism.

1. A flow cell, comprising
a support layer of low-background material;
a film of anodized metal oxide (AMO) material adhered to the support layer; and
a substrate surface comprising one or more patterns imparted in the AMO material, each pattern of the one or more patterns comprising
an array of nanowell features surrounded by interstitial regions of featureless AMO film, wherein
an interior volume of each nanowell feature has an average depth at least equal to a thickness of an interstitial region surrounding each respective feature,
each nanowell feature further comprising
an opening formed in the AMO material, and
a base comprising an exposed surface of low-background material of the support layer, wherein the exposed surface of low-background material is functionalized as a solid support for detection of a biological sample.

2. The flow cell of claim 1, wherein the substrate surface has a length along a y axis and a width along an x axis, and wherein
the one or more patterned comprise two or more at least two patterns, each arranged in lanes along the y axis and separated along the x axis by a noncontiguous masking layer bonded to the AMO film.

3. The flow cell of claim 2, wherein the lanes define two or more channels for flowing a solution containing the biological sample over the at least two patterns.

4. The flow cell of claim 1, wherein the AMO film comprises an anodic alumina oxide (AAO) material.

5. The flow cell of claim 1, wherein the low-background material comprises a glass material.

6. The flow cell of claim 5, wherein each solid support comprises a silanized glass surface.

7. The flow cell of claim 6, wherein each solid support comprises
a polymer layer contiguous with the silanized glass surface; and
a surface chemistry grafted to the polymer layer, wherein the surface chemistry is adapted to interact with the biological sample.

8. The flow cell of claim 7, wherein the biological sample comprises a pool of constituent analytes, and the surface chemistry comprises one or more primers grafted to the polymer layer of each solid support, and wherein
each of the one or more primers is adapted to interact with one or more constituent analytes.

9. The flow cell of claim 1, wherein the openings of the nanowell features are substantially circular.

10. The flow cell of claim 9, wherein an average diameter of the substantially circular openings of the nanowell features is between about 250 nm and 400 nm.

11. The flow cell of claim 1, wherein the openings of the nanowell features are substantially hexagonal.

12. The flow cell of claim 12, wherein an average long diagonal distance of the substantially hexagonal openings is between about 250 nm and 400 nm.

13. The flow cell of claim 1, wherein an average pitch of the openings of the nanowell features is between about 350 nm and 750 nm.

14. The flow cell of claim 1, wherein an average depth of the nanowell features is between about 200 nm and 400 nm.

15. A biopolymeric assay comprising a flow cell functionalized with a surface chemistry on which a biopolymeric sample is immobilized, the flow cell comprising
a support layer of low-background material,
a cladding layer of anodic alumina oxide (AAO) material, and
a substrate surface having one or more patterns etched in the AAO film, each pattern comprising
an array of nanowell features surrounded by interstitial planar regions of the cladding layer, wherein an interior volume of each nanowell has a depth at least equal to a thickness of an interstitial region surrounding each respective nanowell, wherein each well further comprises
an opening formed in the cladding, and,
a bottom opposite the opening, wherein
the bottom and interior of the nanowell are in fluid communication with the substrate surface via the opening, and
the bottom comprises a solid support formed of an exposed surface of the low-background material of the support layer;
the surface chemistry comprising a one or more primers grafted to each solid support of at least a portion the array of nanowell features, each primer comprising a capture moiety adapted to interact with a constituent analyte of a biopolymeric sample; and
the biopolymeric sample comprising a pool of constituent analytes,
wherein at least one constituent analyte of the pool is immobilized on each solid support of at least a portion of the primer-grafted solid supports of the array of nanowell features through interaction with the capture moiety of a respective primer.

16. The biopolymeric assay of claim 15, wherein the biopolymeric sample is a nucleic acid material.

17. The biopolymeric assay of claim 16, wherein the nucleic acid material is DNA and each constituent analyte comprises a DNA fragment.

18. The biopolymeric assay of claim 17, wherein each DNA fragment comprises an adapter sequence of nucleotides and the capture moiety comprises a complementary sequence of nucleotides, wherein
interaction comprises hybridization of the adapter sequence of each of the immobilized constituent analytes with a complementary sequence of a respective capture moiety.

19. A method of detecting a nucleic acid sample comprising providing a flow cell comprising
a support layer of a low-background material,
a film of anodized metal oxide (AMO) material adhered to the support layer, and
a substrate surface comprising one or more patterns etched in the AMO material, each pattern of the one or more patterns comprising
an array of nanowell features surrounded by interstitial regions of featureless AMO film, wherein an interior volume of each nanowell has a depth at least equal to a thickness of a surrounding interstitial region of a respective feature, each nanowell feature further comprising:
an opening formed in the AMO material; and
a bottom opposite the opening, wherein
the bottom and interior of the nanowell are in fluid communication with the substrate surface via the opening, and
the bottom comprises a solid support formed of an exposed surface of the low-background material of the support layer;
grafting a surface chemistry to each solid support of at least a portion of the array of nanowell features, each surface chemistry comprising a one or more primers grafted to a respective solid support, and each primer comprising a capture moiety adapted to interact with a constituent analyte of the nucleic acid sample;
processing the nucleic acid sample into a pool of fragment polynucleotide analytes in solution;
flowing the solution on the substrate surface;
contacting at least a portion of the pool of constituent polynucleotide analytes with at least a portion of the array of nanowell features such that at least one constituent polynucleotide analyte of the portion of the pool is immobilized on one solid support of the portion of the array; and
detecting a subject analyte of the immobilized constituent analytes.

20. The method of claim 19, further comprises providing an optical detection system comprising
an excitation source;
one or more optical sensors; and
a signal processor, wherein
detection of a subject analyte comprises
irradiating, by the excitation source, the subject analyte with an excitation light, wherein the irradiation of the subject analyte causes emission of an optical signal from the subject analyte;
detecting, by the one or more optic sensor, the optical signal;
obtaining from the optical signal, by the signal processor, data indicative of a characteristic of the subject analyte.

21. The method of claim 20, wherein the characteristic is a base type of a nucleotide constituent of the subject analyte.
